(19) United States
(12) Patent Application Publication
(10) Pub. No.: US 2015/0031024 A1
(43) Pub. Date: Jan. 29, 2015

(54) ENZYMATIC PREPARATION OF 10 BASE TO 50 KB DOUBLE-STRAND DNA REAGENT FOR SEQUENCING WITH A NANOPORE-POLYMERASE SEQUENCING DEVICE

(71) Applicants: Hugh E. Olsen, Santa Cruz, CA (US); Mark A. Akeson, Santa Cruz, CA (US)

(72) Inventors: Hugh E. Olsen, Santa Cruz, CA (US); Mark A. Akeson, Santa Cruz, CA (US)

(73) Assignee: THE REGENTS OF THE UNIVERSITY OF CALIFORNIA, Oakland, CA (US)

(21) Appl. No.: 14/234,141

(22) PCT Filed: Jul. 30, 2012
(86) PCT No.: PCT/US12/48906

§ 371 Date: Aug. 25, 2014

Related U.S. Application Data
(60) Provisional application No. 61/574,232, filed on Jul. 30, 2011.

(51) Int. Cl. C12Q 1/68

(52) U.S. Cl. CPC C12Q 1/6869; C12Q 1/6876
USPC 435/6.11; 435/287.2

(57) ABSTRACT

The invention herein disclosed provides for devices, reagents, and methods that can detect and control an individual polymer in a mixture is acted upon by another compound, for example, an enzyme, in a nanopore. Of particular note is the use of reagents to rapidly sequence a polynucleotide. The invention is of particular use in the fields of forensic biology, molecular biology, structural biology, cell biology, molecular switches, molecular circuits, and molecular computational devices, and the manufacture thereof.

The present application claims priority to and benefits of U.S. Provisional Patent Application Ser. No. 61/574,232 entitled “Enzymatic Preparation Of 10 Base To 50 Kb Double-Strand DNA Reagent For Sequencing With A Nanopore-Polymerase Sequencing Device”, filed 30 Jul. 2011, which is herein incorporated by reference in its entirety for all purposes.

This invention was made partly using funds from the National Human Genome Research Institute grant number 5RC2NG00553-02. The Federal Government has certain rights to this invention.

### FIELD OF THE INVENTION

The invention herein disclosed provides for devices and methods that can regulate the time at which an individual polymer in a mixture is acted upon by another compound, for example, an enzyme. The invention is of particular use in the fields of molecular biology, structural biology, cell biology, molecular switches, molecular circuits, and molecular computational devices, and the manufacture thereof. The invention also relates to methods of using the compositions to diagnose whether a subject is susceptible to cancer, autoimmune diseases, cell cycle disorders, or other disorders.

### BACKGROUND

The invention relates to the field of compositions and reagents, methods, and apparatus for characterizing polynucleotides and other polymers.

Determining the nucleotide sequence of DNA and RNA in a rapid manner is a major goal of researchers in biotechnology, especially for projects seeking to obtain the sequence of entire genomes of organisms. In addition, rapidly determining the sequence of a polynucleotide is important for identifying genetic mutations and polymorphisms in individuals and populations of individuals.

Nanopore sequencing is one method of rapidly determining the sequence of polynucleotide molecules. Nanopore sequencing is based on the property of physically sensing the individual nucleotides (or physical changes in the environment of the nucleotides (that is, for example, an electric current)) within an individual polynucleotide (for example, DNA and RNA) as it traverses through a nanopore aperture. In principle, the sequence of a polynucleotide can be determined from a single molecule. However, in practice, it is preferred that a polynucleotide sequence be determined from a statistical average of data obtained from multiple passages of the same molecule or the passage of multiple molecules having the same polynucleotide sequence. The use of membrane channels to characterize polynucleotides as the molecules pass through the small ion channels has been studied by Kasianowicz et al. (Proc. Natl. Acad. Sci. USA. 93: 13770-13773, 1996, incorporated herein by reference) by using an electric field to force single stranded RNA and DNA molecules through a 1.5 nanometer diameter nanopore aperture (for example, an ion channel) in a lipid bilayer membrane. The diameter of the nanopore aperture permitted only a single strand of a polynucleotide to traverse the nanopore aperture at any given time. As the polynucleotide traversed the nanopore aperture, the polynucleotide partially blocked the nanopore aperture, resulting in a transient decrease of ionic current. Since the length of the decrease in current is directly proportional to the length of the polynucleotide, Kasianowicz et al. (1996) were able to determine experimentally lengths of polynucleotides by measuring changes in the ionic current.

Baldarelli et al. (U.S. Pat. No. 6,015,714) and Church et al. (U.S. Pat. No. 5,795,782) describe the use of nanopores to characterize polynucleotides including DNA and RNA molecules on a monomer by monomer basis. In particular, Baldarelli et al. characterized and sequenced the polynucleotides by passing a polynucleotide through the nanopore aperture. The nanopore aperture is imbedded in a structure or an interface, which separates two media. As the polynucleotide passes through the nanopore aperture, the polynucleotide alters an ionic current by blocking the nanopore aperture. As the individual nucleotides pass through the nanopore aperture, each base/nucleotide alters the ionic current in a manner that allows the identification of the nucleotide transiently blocking the nanopore aperture, thereby allowing one to characterize the nucleotide composition of the polynucleotide and perhaps determine the nucleotide sequence of the polynucleotide.

One disadvantage of previous nanopore analysis techniques is controlling the rate at which the target polynucleotide is analyzed. As described by Kasianowicz, et al. (1996), nanopore analysis is a useful method for performing length determinations of polynucleotides. However, the translocation rate is nucleotide composition dependent and can range between 10⁵ to 10⁷ nucleotides per second under the measurement conditions outlined by Kasianowicz et al. (1996). Therefore, the correlation between any given polynucleotide's length and its translocation time is not straightforward. It is also anticipated that a higher degree of resolution with regard to both the composition and spatial relationship between nucleotide units within a polynucleotide can be obtained if the translocation rate is substantially reduced.

Recently, the properties of DNA or RNA molecules bound to nucleic acid processing enzymes have been analyzed at a nanopore orifice. The complexes studied include those of single-stranded DNA with Escherichia coli Exonuclease I (Hornblower, B.; Coombs, A.; Whitaker, R. D.; Kolomeisky, A.; Picone, S. J.; Meller, A.; Akeson, M. Nat. Methods. 2007, 4, 315-317), RNA with the bacteriophage phi8 ATPase (Astier, Y.; Kainov, D. E.; Bayley, H.; Tuma, R.; Howorka, S. Chemphyschem. 2007, 8, 2189-2194), and primer/template DNA substrates bound to the 3′-5′-exonuclease deficient versions of two A-family DNA polymerases, the Klenow fragment of E. coli DNA polymerase (KF(exo-)) and bacteriophage T7 DNA polymerase (T7DNAP(exo-)) (Benner, S.; Chen, R. J.; Wilson, N. A.; Abu-Shumays, R.; Hurt, N.; Lieberman, K. R.; Deamer, D. W.; Dunbar, W. B.; Akeson, M. Nat. Nanotechnol. 2007, 2, 718-724; Cockroft, S. L.; Chu, J.; Amorin, M.; Ghadiri, M. R. J. Am. Chem. Soc. 2008, 130, 818-820; Gyarfas, B.; Olasagasti, F.; Benner, S.; Garalde, D.; Lieberman, K. R.; Akeson, M. ACS. Nano. 2009, 3, 1457-1466; Hurt, N.; Wang, H.; Akeson, M.; Lieberman, K. R. J. Am. Chem. Soc. 2009, 131, 3772-3778; Wilson, N. A.; Abu-Shumays, R.; Gyarfas, B.; Wang, H.; Lieberman, K. R.; Akeson, M.; Dunbar, W. B. ACS. Nano. 2009, 3, 995-1003. We have demonstrated that T7DNAP(exo-) could replicate and advance a DNA template held in the α-hemolysin (α-HL) nanopore against an 80 mV applied potential (Olasagasti, F.; Lieberman, K. R.; Benner, S.; Cherf, G. M.; Dahl, J. M.; Deamer, D. W.; Akeson, M., Nat. Nanotechnol. 2010, 5(11): 789-806, doi:10.1038/nnano.2010.2177). However, due to the low stability of the T7DNAP(exo-)-DNA complex under load, diminished signal to noise ratio at 80 mV potential, and the high turnover rate of the polymerase, it was difficult to detect ionic current steps that reported more than three sequential nucleotide additions during replication. International Patent Application No. PCT/US2008/004467 and related U.S. patent application Ser. Nos. 12/080,684 and 12/459,059 disclose a number of technologies that comprise α-hemolysin nanopores coupled with several exemplary DNA polymerases that may be used with the technologies disclosed herein.

There is currently a need to provide compositions and methods that can be used in characterization of polymers, including polynucleotides and polypeptides, as well as diagnosis and prognosis of diseases and disorders. There is also a need in the art to provide systems and methods that can detect single nucleotides in a timeframe that can be used to distinguish not only between individual nucleotides in a polynucleotide but also the chemical characteristics of the individual nucleotide. In particular there is also a need to provide compositions that are tolerant in vitro to elevated concentrations of salts.

### BRIEF DESCRIPTION OF THE INVENTION

In one embodiment, the invention contemplates a reagent for determining the nucleotide sequence of a target polynucleotide in a sample, the reagent comprising a first reagent component, a second reagent component, a third reagent component, and a DNA modifying enzyme, (i) wherein the first reagent component comprises a 1^{st} polynucleotide partial duplex, the 1^{st} polynucleotide partial duplex comprising a polynucleotide duplex and a first polynucleotide single strand and a second polynucleotide single strand, the polynucleotide duplex comprising a blocking oligomer and a loading oligomer, wherein a portion of the blocking oligomer comprises a nucleotide sequence that is the complement of a portion of the loading oligomer, wherein a portion of the blocking oligomer comprises a nucleotide sequence that is not the complement of a portion of the loading oligomer, wherein the blocking oligomer is annealed to the loading oligomer between the complementary portion of the blocking oligomer and the complementary portion of the loading oligomer thereby creating a proximal portion of the polynucleotide partial duplex, whereby the portion of the blocking oligomer that is not the complement of the loading oligomer is the first single strand and the portion of the loading oligomer that is not the complement of the blocking oligomer is the second single strand, and wherein the blocking oligomer first single strand and the loading oligomer second single strand comprise a distal portion of the polynucleotide partial duplex; (ii) wherein the second reagent component comprises a 2^{nd} polynucleotide partial duplex, the 2^{nd} polynucleotide partial duplex comprising a single polynucleotide, wherein a first portion of the single polynucleotide is substantially the complement of a second portion of the polynucleotide, and wherein the 2^{nd} polynucleotide partial duplex comprises the first portion of the single polynucleotide is annealed to the second portion of the polynucleotide thereby creating a hairpin structure, the hairpin structure comprising a hairpin loop, a hairpin stem, the hairpin stem further comprising at least one acridine nucleotide residue, and third portion of the polynucleotide that is not annealed to either portion of the polynucleotide, the third portion further comprising a restriction endonuclease site; (iii) wherein the third reagent component comprises the target polynucleotide, the target polynucleotide substantially comprising a double strand polynucleotide and a first end and a second end, wherein the first end comprises a first strand that is the complement of the blocking oligomer and a second strand that is the complement of the loading oligomer, wherein the second end comprises a polynucleotide sequence that is the complement of the hairpin structure; and wherein the first and second and third reagent components are at an equimolar ratio. In a preferred embodiment the DNA modifying enzyme is a DNA ligase.

In another embodiment, the invention also contemplates a reagent for determining the nucleotide sequence of a target polynucleotide in a sample comprising the first reagent. In an alternative embodiment, the invention also contemplates a reagent for determining the nucleotide sequence of a target polynucleotide in a sample comprising the second reagent. In one preferred embodiment, the reagent for determining the nucleotide sequence of a target polynucleotide in a sample, comprising the first reagent, the second reagent, or both the first reagent and the second reagent, further comprises a composition selected from the group consisting of, at least one dNTP molecule, a metal ion co-factor, diacylglycerol, phosphatidylserine, eicosinoids, retinoic acid, calciferol, ascorbic acid, neuropeptides, enkephalins, endorphins, 4-aminobutyrate (GABA), 5-hydroxytryptamine (5-HT), catecholamines, acetyl CoA, S-adenosylmethionine, and any other biological activator. In a more preferred embodiment, the cofactor is selected from the group consisting of Mg²⁺, Mn²⁺, Ca²⁺, ATP, NAD⁺, NADP⁺, and any other biological cofactor.

The invention also contemplates a system for determining the nucleotide sequence of a polynucleotide in a sample, the system comprising an electrical source, an anode, a cathode, a cis chamber, a trans chamber, wherein the cis and the trans chambers are separated by a thin film, the thin film having a plurality of apertures, wherein each aperture is between about 0.25 nm and about 4 nm in diameter, a conducting solvent, a processive DNA modifying enzyme, a plurality of dNTP molecules, a metal ion co-factor, and the reagent disclosed herein.

The invention also contemplates an apparatus for determining the nucleotide sequence of a polynucleotide in a sample, the apparatus comprising an electrical source, an anode, a cathode, a cis chamber, a trans chamber, wherein the cis and the trans chambers are separated by a thin film, the thin film having a plurality of apertures (pores), wherein each aperture (pore) is between about 0.25 nm and about 4 nm in diameter, a conducting solvent, a processive DNA modifying enzyme, a plurality of dNTP molecules, a metal ion co-factor, and the reagent disclosed herein.

The invention also contemplates a device for determining the nucleotide sequence of a polynucleotide in a sample, the device comprising an electrical source, an anode, a cathode, a cis chamber, a trans chamber, wherein the cis and the trans chambers are separated by a thin film, the thin film having a plurality of apertures (pores), wherein each aperture (pore) is between about 0.25 nm and about 4 nm in diameter, a conducting solvent, a processive DNA modifying enzyme, a plurality of dNTP molecules, a metal ion co-factor, and the reagent disclosed herein.

The invention also contemplates a method for determining the nucleotide sequence of a polynucleotide in a sample, the method comprising the steps of: providing two separate adjacent chambers comprising a liquid medium, an interface between the two chambers, the interface having an aperture so dimensioned as to allow sequential monomer-by-monomer passage from the cis-side of the channel to the trans-side of the channel of only one polynucleotide strand at a time; providing the reagent disclosed herein; introducing the reagent into one of the two chambers; allowing the processive DNA-modifying enzyme to bind to the polynucleotide; applying a potential difference between the two chambers, thereby creating a first polarity, the first polarity causing the single-stranded portion of the polynucleotide to transpose through the aperture to the trans-side; introducing the enzyme into the same chamber; allowing the enzyme to bind to the polynucleotide; measuring the electrical current through the channel thereby detecting a nucleotide base in the polynucleotide; decreasing the potential difference a first time; allowing the single-stranded portion of the polynucleotide to transpose through the aperture; measuring the change in electrical current; increasing the potential difference; measuring the electrical current through the channel, thereby detecting a particular nucleotide base positioned at the aperture; repeating any one of the steps, thereby determining the nucleotide sequence of the polynucleotide.

In a more preferred embodiment the method further comprises a step of adding at least one species of dNTP molecule. In another more preferred embodiment the system, apparatus, device, or method further comprises at least one species of dNTP molecule.

In a yet more preferred embodiment the concentration of one dNTP molecule is at least two orders of magnitude lower than the concentration of the other dNTP molecules.

In a more preferred embodiment the system, apparatus, device, or method further comprises an ammeter.

In a more preferred embodiment the aperture diameter is about 2 nm.

In a more preferred embodiment the conducting solvent is an aqueous solvent. In an alternative more preferred embodiment the conducting solvent is a non-aqueous solvent.

In a preferred embodiment, the processive DNA modifying enzyme is a DNA polymerase. In another preferred embodiment, the processive DNA modifying enzyme is selected from the group consisting of phi29 DNA polymerase, T7 DNA polymerase, His 1 DNA polymerase, and His 2 DNA polymerase, Bacillus phage M2 DNA polymerase, Streptococcus phage CP1 DNA polymerase, enterobacter phage PRD1 DNA polymerase, and variants thereof. In another preferred embodiment, the variant of the DNA modifying enzyme has at least 85% amino acid identity with the wild-type DNA modifying enzyme.

In one embodiment the invention contemplates use of the reagent disclosed herein to control the movement of a target polynucleotide through a pore. In a preferred embodiment, the use of the reagent to control the movement of a target polynucleotide through a pore, wherein the use of the reagent further comprises (i) providing the reagent as disclosed herein adjacent to a pore in a DNA sequencing device, (ii) positioning a DNA Polymerase in relationship to a nanopore and a target polynucleotide to be sequenced thereby allowing the DNA polymerase to function as a molecular motor and thereby providing single base resolution movement of the target polynucleotide through the pore in the DNA sequencing device, (iii) whereby the position of the DNA Polymerase in relationship to a nanopore and a target polynucleotide generates a diagnostic signal in the DNA sequencing device thereby indicating the target polynucleotide and DNA polymerase are in the correct position to begin sequencing the target polynucleotide in the DNA sequencing device, (iv) generating signals confirming steps in the sequencing of individual bases, including the beginning, the middle and then end of the target polynucleotide, and (v) repeating (i), (ii), (iii), and (iv).

In another embodiment the invention contemplates a kit for sequencing a target polynucleotide comprising (a) a pore and (b) the reagent as disclosed herein.

In another embodiment the invention contemplates an analysis apparatus for sequencing target polynucleotides in a sample, comprising a plurality of pores and the reagent as disclosed herein.

In a preferred embodiment the analysis apparatus comprises: a sensor device that is capable of supporting the plurality of pores and being operable to perform polynucleotide sequencing using the pores, polymerases and the reagent disclosed herein; at least one reservoir for holding material for performing the sequencing; a fluidics system configured to controllably supply material from the at least one reservoir to the sensor device; and a plurality of containers for receiving respective samples, the fluidics system being configured to supply the samples selectively from the containers to the sensor device.

The invention also provides a kit for sequencing a target polynucleotide comprising (a) a pore and (b) a Phi29 DNA polymerase.

The invention also provides an analysis apparatus for sequencing target polynucleotides in a sample, comprising a plurality of pores and a plurality of Phi29 DNA polymerases.

The invention also provides a system for determining the nucleotide sequence of a polynucleotide in a sample, the system comprising an electrical source, an anode, a cathode, a cis chamber, a trans chamber, wherein the cis and the trans chambers are separated by a thin film, the thin film having a plurality of apertures (pores), wherein each aperture (pore) is between about 0.25 nm and about 4 nm in diameter, a conducting solvent, a processive DNA modifying enzyme, a plurality of dNTP molecules, the reagent disclosed herein, and a metal ion co-factor.

In one embodiment, the system further comprises at least one species of dNTP molecule. In another embodiment, the system further comprises an ammeter. In one preferred embodiment, the aperture diameter is about 2 nm. In another embodiment, the conducting solvent is an aqueous solvent. In an alternative embodiment the conducting solvent is a non-aqueous solvent. In another embodiment, the processive DNA modifying enzyme is a DNA polymerase. In another embodiment, the processive DNA modifying enzyme is tolerant to at least 0.6 M monovalent salt. In another embodiment, the concentration of the monovalent salt is at saturation. In another embodiment, the concentration of the monovalent salt is between 0.6 M and at saturation. In another embodiment, the processive DNA modifying enzyme is isolated from a mesophile or a virus naturally infecting a mesophile. In another embodiment, the processive DNA modifying enzyme is isolated from a halophile or a virus naturally infecting a halophile. In another embodiment, the processive DNA modifying enzyme is isolated from an extreme halophile or a virus naturally infecting an extreme halophile.

In another embodiment, the processive DNA modifying enzyme is selected from a bacterium from the group consisting of Haloferax, Halogeometricum, Halococcus, Haloterrigena, Halorubrum, Haloarcula, Halobacterium, Salinivibrio costicola. Halomonas elongata, Halomonas israeiensis, Salinibacter rube, Dunaliella saliva, Staphylococcus aureus, Actinopolyspora halophila, Marinococcus halophilus, and S. costicola. In another embodiment, the processive DNA modifying enzyme is selected from the group consisting of phi29 DNA polymerase, T7 DNA polymerase, His 1 DNA polymerase, and His 2 DNA polymerase, Bacillus phage M2 DNA polymerase, Streptococcus phage CP1 DNA polymerase, enterobacter phage PRD1 DNA polymerase, and variants thereof.

In a preferred embodiment, the processive DNA modifying enzyme is phi29 DNA polymerase.

In another embodiment, the processive DNA modifying enzyme is from a moderate halophile, wherein the moderate halophile is selected from the group consisting of Pseudomonas, Flavobacterium, Spirochaeta, Salinivibrio, Arhodomonas, and Dichotomicrobium.

In another embodiment, the invention provides an apparatus for determining the nucleotide sequence of a polynucleotide in a sample, the apparatus comprising an electrical source, an anode, a cathode, a cis chamber, a trans chamber, wherein the cis and the trans chambers are separated by a thin film, the thin film having a plurality of apertures (or pores), wherein each aperture (or pore) is between about 0.25 nm and about 4 nm in diameter, a conducting solvent, a processive DNA modifying enzyme, a plurality of dNTP molecules, the reagent disclosed herein, and a metal ion co-factor.

In one embodiment, the apparatus further comprises at least one species of dNTP molecule. In another embodiment, the apparatus further comprises an ammeter. In one preferred embodiment, the aperture diameter is about 2 nm. In another embodiment, the conducting solvent is an aqueous solvent. In an alternative embodiment the conducting solvent is a non-aqueous solvent. In another embodiment, the processive DNA modifying enzyme is a DNA polymerase. In another embodiment, the processive DNA modifying enzyme is tolerant to at least 0.6 M monovalent salt. In another embodiment, the concentration of the monovalent salt is at saturation. In another embodiment, the concentration of the monovalent salt is between 0.6 M and at saturation. In another embodiment, the processive DNA modifying enzyme is isolated from a mesophile or a virus naturally infecting a mesophile. In another embodiment, the processive DNA modifying enzyme is isolated from a halophile or a virus naturally infecting a halophile. In another embodiment, the processive DNA modifying enzyme is isolated from an extreme halophile or a virus naturally infecting an extreme halophile.

In another embodiment, the processive DNA modifying enzyme is selected from a bacterium from the group consisting of Haloferax, Halogeometricum, Halococcus, Haloterrigena, Halorubrum, Haloarcula, Halobacterium, Salinivibrio costicola, Halomonas elongata, Halomonas isralensis, Salinibacter rube, Dunaliella saliva, Staphylococcus aureus, Actinopolyspora halophila, Marinococcus halophilus, and S. costicola. In another embodiment, the processive DNA modifying enzyme is selected from the group consisting of phi29 DNA polymerase, T7 DNA polymerase, His 1 DNA polymerase, and His 2 DNA polymerase, Bacillus phage M2 DNA polymerase, Streptococcus phage CP1 DNA polymerase, enterobacter phage PRD1 DNA polymerase, and variants thereof.

In a preferred embodiment, the processive DNA modifying enzyme is phi29 DNA polymerase.

In another embodiment, the processive DNA modifying enzyme is from a moderate halophile, wherein the moderate halophile is selected from the group consisting of Pseudomonas, Flavobacterium, Spirochaeta, Salinivibrio, Arhodomonas, and Dichotomicrobiuin.

In a yet other embodiment, the invention provides a device for determining the nucleotide sequence of a polynucleotide in a sample, the device comprising an electrical source, an anode, a cathode, a cis chamber, a trans chamber, wherein the cis and the trans chambers are separated by a thin film, the thin film having a plurality of apertures (or pores), wherein each aperture (or pore) is between about 0.25 nm and about 4 nm in diameter, a conducting solvent, a processive DNA modifying enzyme, a plurality of dNTP molecules, the reagent disclosed herein, and a metal ion co-factor.

In one embodiment, the device further comprises at least one species of dNTP molecule. In another embodiment, the device further comprises an ammeter. In one preferred embodiment, the aperture diameter is about 2 nm. In another embodiment, the conducting solvent is an aqueous solvent. In an alternative embodiment the conducting solvent is a non-aqueous solvent. In another embodiment, the processive DNA modifying enzyme is a DNA polymerase. In another embodiment, the processive DNA modifying enzyme is tolerant to at least 0.6 M monovalent salt. In another embodiment, the concentration of the monovalent salt is at saturation. In another embodiment, the concentration of the monovalent salt is between 0.6 M and at saturation. In another embodiment, the processive DNA modifying enzyme is isolated from a mesophile or a virus naturally infecting a mesophile. In another embodiment, the processive DNA modifying enzyme is isolated from a halophile or a virus naturally infecting a halophile. In another embodiment, the processive DNA modifying enzyme is isolated from an extreme halophile or a virus naturally infecting an extreme halophile.

In another embodiment, the processive DNA modifying enzyme is selected from a bacterium from the group consisting of Haloferax, Halogeometricum, Halococcus, Haloterrigena, Halorubrum, Haloarcula, Halobacterium, Salinivibrio costicola, Halomonas elongata, Halomonas israelensis, Salinibacter rube, Dunaliella salina, Staphylococcus aureus, Actinopolyspora halophila, Marinococcus halophilus, and S. costicola. In another embodiment, the processive DNA modifying enzyme is selected from the group consisting of phi29 DNA polymerase, T7 DNA polymerase, His 1 DNA polymerase, and His 2 DNA polymerase, Bacillus phage M2 DNA polymerase, Streptococcus phage CP1 DNA polymerase, enterobacter phage PRD1 DNA polymerase, and variants thereof.

In a preferred embodiment, the processive DNA modifying enzyme is phi29 DNA polymerase.

In another embodiment, the processive DNA modifying enzyme is from a moderate halophile, wherein the moderate halophile is selected from the group consisting of Pseudomonas, Flavobacterium, Spirochaeta, Salinivibrio, Arhodomonas, and Dichotomicrobium.

In another embodiment, the invention provides a method for sequencing a polynucleotide, the method further comprising a step of including a blocking oligoiner. In a preferred embodiment, the blocking oligomer comprises at least 15 nucleotides. In another preferred embodiment, the blocking oligomer comprises at least 20 nucleotides. In another preferred embodiment, the blocking oligoiner comprises at least 25 nucleotides. In another preferred embodiment, the blocking oligomer comprises at least 30 nucleotides. In another preferred embodiment, the blocking oligomer comprises at least 35 nucleotides, in another preferred embodiment, the blocking oligomer comprises at least 40 nucleotides. In another preferred embodiment, the blocking oligomer comprises at least 45 nucleotides. In another preferred embodiment, the blocking, oligomer comprises at least 50 nucleotides. In an alternative embodiment the blocking oligomer is selected from the group consisting of a 10-mer, a 15-mer, a 20-mer, a 25-mer, a 30-mer, a 31-mer, a 32-mer, a 33-mer, a 34-mer, a 35-mer, a 36-mer, a 37-mer, a 38-mer, a 39-mer, a 40-mer, a 50-mer, or any number of nucleotides therebetween, it may also be desirable to provide a blocking oligoiner having more than 50 nucleotides.

In another embodiment the invention provides a method of sequencing a polynucleotide, wherein the polynucleotide has a size in the range of between 10 nucleotides to 50 thousand nucleotides. The number of nucleotides in the polynucleotide can be 10, 15, 20, 25, 30, 35, 40, 45, 50, 75, 100, 150, 200, 250, 300, 350, 400, 450, 500, 750, 1,000, 1,500, 2,000, 2,500, 3,000, 3,500, 4,000, 4,500, 5,000, 10, 000, 15,000, 20, 000, 30, 000, 40,000, 50,000 or any number therebetween. It may also be desirable to sequence polynucleotides in excess of 50,000 nucleotides.

The invention provides thin film devices, systems, reagents, and methods for using the same. The subject devices or systems comprise cis and trans chambers connected by an electrical communication means. The cis and trans chambers are separated by a thin film comprising at least one pore or channel. In one preferred embodiment, the thin film comprises a compound having a hydrophobic domain and a hydrophilic domain. In a more preferred embodiment, the thin film comprises a phospholipid. The devices or systems further comprise a means for applying an electric field between the cis and the trans chambers. The pore or channel is shaped and sized having dimensions suitable for passaging a polymer. In one preferred embodiment the pore or channel accommodates a part but not all of the polymer. In one other preferred embodiment, the polymer is a polynucleotide. In an alternative preferred embodiment, the polymer is a polypeptide. Other polymers provided by the invention include polypeptides, phospholipids, polysaccharides, and polyketides.

In one embodiment, the thin film further comprises a compound having a binding affinity for the polymer. In one preferred embodiment the binding affinity (Kₐ) is at least 10⁶ l/mole. In a more preferred embodiment the Kₐ is at least 10⁸ l/mole. In yet another preferred embodiment the compound is adjacent to at least one pore. In a more preferred embodiment the compound is a channel. In a yet more preferred embodiment the channel has biological activity. In a most preferred embodiment, the compound comprises the pore.

In another embodiment the pore is sized and shaped to allow passage of an activator, wherein the activator is selected from the group consisting of ATP, NAD⁺, NADP⁺, diacylglycerol, phosphatidylserine, eicosinoids, retinoic acid, calciferol, ascorbic acid, neuropeptides, enkephalins, endorphins, 4-aminobutyrate (GABA), 5-hydroxytryptamine (5-HT), catecholamines, acetyl CoA, S-adenosylmethionine, and any other biological activator.

In yet another embodiment the pore is sized and shaped to allow passage of a cofactor, wherein the cofactor is selected from the group consisting of Mg²⁺, Mn²⁺, Ca²⁺, ATP, NAD⁺, NADP⁺, and any other biological cofactor.

In a preferred embodiment the pore or channel is a pore molecule or a channel molecule and comprises a biological molecule, or a synthetic modified molecule, or altered biological molecule, or a combination thereof. Such biological molecules are, for example, but not limited to, an ion channel, a nucleoside channel, a peptide channel, a sugar transporter, a synaptic channel, a transmembrane receptor, such as GPCRs and the like, a nuclear pore, synthetic variants, chimeric variants, or the like. In one preferred embodiment the biological molecule is α-hemolysin.

In an alternative, the compound comprises non-enzyme biological activity. The compound having non-enzyme biological activity can be, for example, but not limited to, proteins, peptides, antibodies, antigens, nucleic acids, peptide nucleic acids (PNAs), locked nucleic acids (LNAs), morpholinos, sugars, lipids, glycophosphoinositols, lipopolysaccharides or the like. The compound can have antigenic activity. The compound can have selective binding properties whereby the polymer binds to the compound under a particular controlled environmental condition, but not when the environmental conditions are changed. Such conditions can be, for example, but not limited to, change in [H⁺], change in environmental temperature, change in stringency, change in hydrophobicity, change in hydrophilicity, or the like.

In another embodiment, the invention provides a compound, wherein the compound further comprises a linker molecule, the linker molecule selected from the group consisting of a thiol group, a sulfide group, a phosphate group, a sulfate group, a cyano group, a piperidine group, an Fmoc group, and a Boc group.

In one embodiment the thin film comprises a plurality of pores. In one embodiment the device comprises a plurality of electrodes.

### Polynucleotides

In another embodiment, the invention provides a method for controlling binding of an enzyme to a partially double-stranded polynucleotide complex, the method comprising: providing two separate, adjacent pools of a medium and an interface between the two pools, the interface having a channel so dimensioned as to allow sequential monomer-by-monomer passage from one pool to the other pool of only one polynucleotide at a time; providing an enzyme having binding activity to a partially double-stranded polynucleotide complex; providing a polynucleotide complex comprising a first polynucleotide and a second polynucleotide, wherein a portion of the polynucleotide complex is double-stranded, and wherein the first polynucleotide further comprises a moiety that is incompatible with the second polynucleotide; introducing the polynucleotide complex into one of the two pools; introducing the enzyme into one of the two pools; applying a potential difference between the two pools, thereby creating a first polarity; reversing the potential difference a first time, thereby creating a second polarity; reversing the potential difference a second time to create the first polarity, thereby controlling the binding of the enzyme to the partially double-stranded polynucleotide complex. In a preferred embodiment, the medium is electrically conductive. In a more preferred embodiment, the medium is an aqueous solution. In a preferred embodiment, the moiety is selected from the group consisting of a peptide nucleic acid, a 2′-O-methyl group, a fluorescent compound, a derivatized nucleotide, and a nucleotide isomer. In another preferred embodiment, the method further comprises the steps of measuring the electrical current between the two pools; comparing the electrical current value obtained at the first time the first polarity was induced with the electrical current value obtained at the time the second time the first polarity was induced. In another preferred embodiment the method further comprises the steps of measuring the electrical current between the two pools; comparing the electrical current value obtained at the first time the first polarity was induced with the electrical current value obtained at a later time. In a more preferred embodiment, the enzyme is selected from the group consisting of DNA polymerase, RNA polymerase, endonuclease, exonuclease, DNA ligase, DNase, uracil-DNA glycosidase, kinase, phosphatase, methylase, and acetylase. In another alternative embodiment, the method further comprises the steps of providing at least one reagent that initiates enzyme activity; introducing the reagent to the pool comprising the polynucleotide complex; and incubating the pool at a suitable temperature. In a more preferred embodiment, the reagent is selected from the group consisting of a deoxyribonucleotide and a cofactor. In a yet more preferred embodiment, the deoxyribonucleotide is introduced into the pool prior to introducing the cofactor. In another still more preferred embodiment, the cofactor is selected from the group consisting of Mg2+, Mn2+, Ca2+, ATP, NAD+, and NADP+. In one embodiment the enzyme is introduced into the same pool as the polynucleotide. In an alternative embodiment, the enzyme is introduced into the opposite pool.

The invention herein disclosed provides for devices, reagents, and methods that can regulate the rate at which an individual polymer in a mixture is acted upon by another compound, for example, an enzyme. The devices, reagents, and methods are also used to determine the nucleotide base sequence of a polynucleotide The invention is of particular use in the fields of molecular biology, structural biology, cell biology, molecular switches, molecular circuits, and molecular computational devices, and the manufacture thereof.

In one embodiment the nanopore system and reagent can control binding of a molecule to a polymer at a rate of between about 5 Hz and 2000 Hz. The nanopore system and reagent can control binding of a molecule to a polymer at, for example, about 5 Hz, at about 10 Hz, at about 15 Hz, at about 20 Hz, at about 25 Hz, at about 30 Hz, at about 35 Hz, at about 40 Hz, at about 45 Hz, at about 50 Hz, at about 55 Hz, at about 60 Hz, at about 65 Hz, at about 70 Hz, at about 75 Hz, at about 80 Hz, at about 85 Hz, at about 90 Hz, at about 95 Hz, at about 100 Hz, at about 110 Hz, at about 120 Hz, at about 125 Hz, at about 130 Hz, at about 140 Hz, at about 150 Hz, at about 160 Hz, at about 170 Hz, at about 175 Hz, at about 180 Hz, at about 190 Hz, at about 200 Hz, at about 250 Hz, at about 300 Hz, at about 350 Hz, at about 400 Hz, at about 450 Hz, at about 500 Hz, at about 550 Hz, at about 600 Hz, at about 700 Hz, at about 750 Hz, at about 800 Hz, at about 850 Hz, at about 900 Hz, at about 950 Hz, at about 1000 Hz, at about 1125 Hz, at about 1150 Hz, at about 1175 Hz, at about 1200 Hz, at about 1250 Hz, at about 1300 Hz, at about 1350 Hz, at about 1400 Hz, at about 1450 Hz, at about 1500 Hz, at about 1550 Hz, at about 1600 Hz, at about 1700 Hz, at about 1750 Hz, at about 1800 Hz, at about 1850 Hz, at about 1900 Hz, at about 950 Hz, and at about 2000 Hz. In a preferred embodiment, the nanopore system can control binding of a molecule to a polymer at a rate of between about 25 Hz and about 250 Hz. In a more preferred embodiment the nanopore system can control binding of a molecule to a polymer at a rate of between about 45 Hz and about 120 Hz. In a most preferred embodiment the nanopore system can control binding of a molecule to a polymer at a rate of about 50 Hz.

The invention also provides thin film devices, reagents, and methods for using the same. The subject devices comprise cis and trans chambers connected by an electrical communication means. The cis and trans chambers are separated by a thin film comprising at least one pore or channel. In one preferred embodiment, the thin film comprises a first compound having a hydrophobic domain and a hydrophilic domain. In a more preferred embodiment, the thin film comprises a phospholipid. The devices further comprise a means for applying an electric field between the cis and the trans chambers. The pore or channel is shaped and sized having dimensions suitable for passaging a polymer. In one preferred embodiment the pore or channel accommodates a part but not all of the polymer. In another preferred embodiment the pore or channel accommodates a monomer part of the polymer but not a dimer part of the polymer. In one other preferred embodiment, the polymer is a polynucleotide. In an alternative preferred embodiment, the polymer is a polypeptide. Other polymers provided by the invention include polypeptides, phospholipids, polysaccharides, and polyketides.

In one embodiment, the thin film further comprises a second compound having a binding affinity for the polymer. In one preferred embodiment the binding affinity (Kₐ) is at least 10⁶ l/mole. In a more preferred embodiment the Kₐ is at least 10⁸ l/mole. In yet another preferred embodiment the compound is adjacent to at least one pore. In a more preferred embodiment the compound is a channel. In a yet more preferred embodiment the channel has biological activity. In a most preferred embodiment, the compound comprises the pore.

In one embodiment the second compound comprises enzyme activity. The enzyme activity can be, for example, but not limited to, enzyme activity of proteases, kinases, phosphatases, hydrolases, oxidoreductases, isomerases, transferases, methylases, acetylases, ligases, lyases, and the like. In a more preferred embodiment the enzyme activity can be enzyme activity of DNA polymerase, RNA polymerase, endonuclease, exonuclease, DNA ligase, DNase, uracil-DNA glycosidase, kinase, phosphatase, methylase, acetylase, or the like.

In one preferred embodiment, the DNA polymerase is isolated from a halophile microorganism. In an alternative preferred embodiment, the DNA polymerase is a naturally-occurring variant of the DNA polymerase isolated from a halophile microorganism. In an alternative preferred embodiment, the DNA polymerase is a synthetic variant of the DNA polymerase isolated from a halophile microorganism. In yet another alternative preferred embodiment, the DNA polymerase is a synthetic composition having the enzyme properties of the DNA polymerase isolated from a halophile microorganism or alternatively, a naturally-occurring variant of the DNA polymerase isolated from a halophile microorganism. In a more preferred embodiment, the halofile microorganism is an extreme halophile microorganism. In another more preferred embodiment the halophile microorganism is a moderate halophile microorganism.

In another preferred embodiment, the halophile microorganism thrives under environmental conditions selected from the group consisting of temperature equal to or greater than 50° C., pressure equal to or greater that 200 kPa, pH equal to or lower than 6.5, pH equal to or greater than 7.5, and salinity equal to or greater than 0.5M. For example, the pressure can be 200 kPa, 225 kPa, 250 kPa, 275 kPa, 300 kPa, or 400 kPa. In another example, the temperature can be 50° C., 55° C., 60° C., 65° C., 70° C., 75° C., 80° C., 85° C., 90° C., 95° C., and 99° C. In another example, the pH can be 2.5, 3.0, 3.5, 4.0, 4.5, 5.0, 5.5, 6.0, 6.5, 7.5, 8.0, 8.5, 9.0, and 9.5.

In an alternative preferred embodiment, the DNA polymerase is isolated from a virus that can infect a halophile microorganism. In an alternative preferred embodiment, the DNA polymerase is a naturally-occurring variant of the DNA polymerase isolated from a virus that can infect a halophile microorganism. In an alternative preferred embodiment, the DNA polymerase is a synthetic variant of the DNA polymerase isolated from a virus that can infect a halophile microorganism. In yet another alternative preferred embodiment, the DNA polymerase is a synthetic composition having the enzyme properties of the DNA polymerase isolated from a virus that can infect a halophile microorganism or alternatively, a naturally-occurring variant of the DNA polymerase isolated from a virus that can infect a halophile microorganism. In a more preferred embodiment, the halofile microorganism is an extreme halophile microorganism. In an alternative embodiment, the virus that can infect a halophile microorganism is infective under environmental conditions selected from the group consisting of temperature equal to or greater than 50° C., pressure equal to or greater that 200 kPa, pH equal to or lower than 6.5, pH equal to or greater than 7.5, and salinity equal to or greater than 0.5M. For example, the temperature can be 50° C., 55° C., 60° C., 65° C., 70° C., 75° C., 80° C., 85° C., 90° C., 95° C., and 99° C. In another example, the pH can be 2.5, 3.0, 3.5, 4.0, 4.5, 5.0, 5.5, 6.0, 6.5, 7.5, 8.0, 8.5, 9.0, and 9.5.

The second compound can have selective binding properties whereby the polymer binds to the second compound under a particular controlled environmental condition, but not when the environmental conditions are changed. Such conditions can be, for example, but not limited to, change in [H⁺], change in environmental temperature, change in stringency, change in hydrophobicity, change in hydrophilicity, or the like.

In another embodiment the pore is sized and shaped to allow passage of an activator, wherein the activator is selected from the group consisting of ATP, NAD⁺, NADP⁺, diacylglycerol, phosphatidylserine, eicosinoids, retinoic acid, calciferol, ascorbic acid, neuropeptides, enkephalins, endorphins, 4-aminobutyrate (GABA), 5-hydroxytryptamine (5-HT), catecholamines, acetyl CoA, S-adenosylmethionine, and any other biological activator.

In yet another embodiment the pore is sized and shaped to allow passage of a cofactor, wherein the cofactor is selected from the group consisting of Mg²⁺, Mn²⁺, Ca²⁺, ATP, NAD⁺, NADP⁺, and any other biological cofactor.

In a preferred embodiment the pore or channel comprises a biological molecule, or a synthetic modified or altered biological molecule. Such biological molecules are, for example, but not limited to, an ion channel, a nucleoside channel, a peptide channel, a sugar transporter, a synaptic channel, a transmembrane receptor, such as GPCRs and the like, a nuclear pore, or the like.

In an alternative, the second compound comprises non-enzyme biological activity. The second compound having non-enzyme biological activity can be, for example, but not limited to, proteins, peptides, antibodies, antigens, nucleic acids, peptide nucleic acids (PNAs), locked nucleic acids (LNAs), morpholinos, sugars, lipids, glycophosphoinositols, lipopolysaccharides or the like.

In another embodiment, the invention provides a third compound, wherein the third compound further comprises a linker molecule, the linker molecule selected from the group consisting of a thiol group, a sulfide group, a phosphate group, a sulfate group, a cyano group, a piperidine group, an Fmoc group, and a Boc group.

In one embodiment the thin film comprises a plurality of pores. In one embodiment the device comprises a plurality of electrodes.

The invention also contemplates a reagent and method of binding DNA polymerase (DNAP) to single-stranded DNA (ss-DNA) and thereby reducing the rate at which the ss-DNA traverses an α-Hemolysin nanopore under a 180 mV applied potential. In a preferred embodiment, single-stranded DNA threads through the DNAP and α-Hemolysin nanopore at a rate near one nucleotide per 1-100 ms. In another embodiment, the rate is from between one nucleotide per 100-1000 ms.

The invention also contemplates a method of using the primer DNA 5′ terminus to protect the template 3′-terminus from digestion by DNA polymerases (DNAP).

The invention also contemplates a method of covalently bonding a C3 (CPG) spacer, followed by an abasic residue on the 3′-terminus and preventing exonucleolytic digestion of the DNA.

The invention also contemplates a method of protecting the primer DNA strand from DNAP function by binding a modified DNA oligomer adjacent to the primer template junction. In a preferred embodiment, DANP binds at the oligomer 5′-terminus and capture of this complex on an α-Hemolysin nanopore with 180 mV applied potential removes the oligomer and places DNAP at the primer terminus, after which DMA replication can take place.

The invention also contemplates a method of using a registry oligomer, preferably a modified DNA oligomer, to control where DNAP binds and sits on the ss-DNA. Capture of these DNAP-DNA complexes on an α-Hemolysin nanopore using a 180 mV applied potential removes the oligomer and allows the s-DNA to translocate through DNAP and the α-Hemolysin.

The invention also contemplates a method wherein phi29 DNAP-bound dsDNA unzips in a nanopore by applied voltage (180 mV). In a preferred embodiment, voltage reduction allows re-zipping of the DNA. Restoring the voltage unzips the DNA again and this allows movement of the DNA back and forth through the nanopore.

The invention also contemplates using a blocking oligomer binding at the DNA primer/transcript junction whereby the oligomer is stripped off when captured on a nanopore, and the DNA is subsequently activated for ratcheting through the nanopore.

The invention also contemplates using shorter blocking oligomers and decreasing the time required to strip the blocking oligomer off the DNA. In a preferred embodiment, this allows activation of DNA molecules for replication on the nanopore faster, and that this increases the throughput of the nanopore for sequencing applications.

The invention also contemplates a method of sequencing a polynucleotide, the method comprising a step of determining the noise level in a signal, the noise level being representative of the identity of the nucleotide inducing the signal compared with the previous nucleotide inducing a signal and the subsequent nucleotide inducing a signal. In a preferred embodiment, the signal is a change in current measured between the two adjacent pools. In a more preferred embodiment, the noise level measured is greater for a nucleotide when the previous nucleotide and/or the subsequent nucleotide are a different nucleotide.

The invention also contemplates a method of sequencing a polynucleotide, the method comprising the step of including a dNTP at lower concentration that other dNTPs thereby reducing the rate of reaction of the DNAP. In one embodiment, the dNTP is at about one order of magnitude lower in concentration that the other dNTPs. In a more preferred embodiment, the dNTP is at about two orders of magnitude lower in concentration that the other dNTPs.

The invention also contemplates a method to properly position a DNA Polymerase (DNAP; Molecular Motor) in relationship to a nanopore and a DNA strand to be sequenced in order to allow the DNA polymerase to function as a molecular motor providing precise single base resolution movement of the DNA through the Nanopore-Polymerase DNA sequencing device. In addition, the method generates a diagnostic signal in the nanopore sequencer indicating the DNA and DNA polymerase (Molecular motor) are in the correct position to begin sequencing in the Nanopore DNA sequencer. Further more, the method generates signals confirming steps in the process of sequencing individual DNA molecules, including the beginning, the middle and then end. Finally, the method provides the opportunity for repeated “reading” of the same DNA strand.

### BRIEF DESCRIPTION OF THE DRAWINGS

FIG. 1 illustrates an exemplary embodiment of the invention showing the ligated DNA sequencing reagent structure.

FIG. 2 illustrates sequential reading and re-reading individual DNA templates on the nanopore using pre-loaded Φ29 DNA polymerase.

### DETAILED DESCRIPTION OF THE INVENTION

The embodiments disclosed in this document are illustrative and exemplary and are not meant to limit the invention. Other embodiments can be utilized and structural changes can be made without departing from the scope of the claims of the present invention. All publications, patents and patent applications cited herein, whether supra or infra, are hereby incorporated by reference in their entirety.

The invention comprises reagents and a method for preparation of DNA of any length for sequencing in the Nanopore-Polymerase DNA sequencing device. The invention further comprises a DNA sequencing reagent that (i) generates signals in a nanopore-polymerase DNA sequencing device confirming that DNA and DNA polymerase are in the correct position to begin sequencing; (ii) generates signals confirming steps in the process of sequencing individual DNA molecules including the beginning and end; (iii) properly positions the DNA polymerase in relationship to the nanopore and the DNA strand to be sequenced in order to allow the DNA polymerase to function as a molecular motor providing precise single base resolution movement of the DNA through the nanopore-polymerase DNA sequencing device.

The invention contemplates addition of duplex features for allowing more rapid sequencing and multiple sequential passes of the same DNA strand through a nanopore-polymerase sequencing device.

As used herein and in the appended claims, the singular forms “a,” “an,” and “the” include plural reference unless the context clearly dictates otherwise. Thus, for example, a reference to “a reagent” includes a plurality of reagents, and a reference to “a signal” is a reference to one or more signals and equivalents thereof, and so forth.

By “polynucleotide” is meant DNA or RNA, including any naturally occurring, synthetic, or modified nucleotide. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, 2-thiocytidine as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

By “transport property” is meant a property measurable during polymer movement with respect to a nanopore. The transport property may be, for example, a function of the solvent, the polymer, a label on the polymer, other solutes (for example, ions), or an interaction between the nanopore and the solvent or polymer.

A “hairpin structure” is defined as an oligonucleotide having a nucleotide sequence that is about 6 to about 10,000 nucleotides in length, the first half of which nucleotide sequence is at least partially complementary to the second part thereof, thereby causing the polynucleotide to fold onto itself, forming a secondary hairpin structure.

“Identity” or “similarity” refers to sequence similarity between two polynucleotide sequences or between two polypeptide sequences, with identity being a more strict comparison. The phrases “percent identity” and “% identity” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences.

The term “incompatible” refers to the chemical property of a molecule whereby two molecules or portions thereof cannot interact with one another, physically, chemically, or both. For example, a portion of a polymer comprising nucleotides can be incompatible with a portion of a polymer comprising nucleotides and another chemical moiety, such as for example, a peptide nucleic acid, a 2′-O-methyl group, a fluorescent compound, a derivatized nucleotide, a nucleotide isomer, or the like. In another example, a portion of a polymer comprising amino acid residues can be incompatible with a portion of a polymer comprising amino acid residues and another chemical moiety, such as, for example, a sulfate group, a phosphate group, an acetyl group, a cyano group, a piperidine group, a fluorescent group, a sialic acid group, a mannose group, or the like.

“Alignment” refers to a number of DNA or amino acid sequences aligned by lengthwise comparison so that components in common (such as nucleotide bases or amino acid residues) may be visually and readily identified. The fraction or percentage of components in common is related to the homology or identity between the sequences. Alignments may be used to identify conserved domains and relatedness within these domains. An alignment may suitably be determined by means of computer programs known in the art, such as MACVECTOR software (1999) (Accelrys, Inc., San Diego, Calif.).

The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present invention may be, for example, variants of the disclosed polynucleotide sequences, including allelic or splice variants, or sequences that encode orthologs or paralogs of presently disclosed polypeptides. Polynucleotide hybridization methods are disclosed in detail by Kashima et al. (1985) Nature 313: 402-404, and Sambrook et al. (1989) Molecular Cloning: A Laboratory Manual, 2nd Ed., Cold Spring Harbor Laboratory, Cold Spring Harbor, N.Y. (“Sambrook”); and by Haymes et al., “Nucleic Acid Hybridization: A Practical Approach”, IRL Press, Washington, D.C. (1985), which references are incorporated herein by reference.

In general, stringency is determined by the incubation temperature, ionic strength of the solution, and concentration of denaturing agents (for example, formamide) used in a hybridization and washing procedure (for a more detailed description of establishing and determining stringency, see below). The degree to which two nucleic acids hybridize under various conditions of stringency is correlated with the extent of their similarity. Thus, similar polynucleotide sequences from a variety of sources, such as within an organism's genome (as in the case of paralogs) or from another organism (as in the case of orthologs) that may perform similar functions can be isolated on the basis of their ability to hybridize with known peptide-encoding sequences. Numerous variations are possible in the conditions and means by which polynucleotide hybridization can be performed to isolate sequences having similarity to sequences known in the art and are not limited to those explicitly disclosed herein. Such an approach may be used to isolate polynucleotide sequences having various degrees of similarity with disclosed sequences, such as, for example, sequences having 60% identity, or more preferably greater than about 70% identity, most preferably 72% or greater identity with disclosed sequences, the resulting sequence having biological activity.

### Reagents of the Invention

In one embodiment the invention comprises three reagent components mixed together in one step to create a DNA reagent for sequencing DNA in a Nanopore-Polymerase Sequencing Device.

Reagent component 1 (or first reagent): A partial duplex consisting of a blocking oligomer annealed with a loading oligomer. The two oligomers are complementary and annealed at one end but not the other end, which contains “two free tails”. The two free tails specifically allow the loading of the DNA strand to be sequenced onto the nanopore (loading oligomer) and permit proper orientation of the polymerase to begin sequencing (blocking oligomer). The annealed duplex end of the blocking oligomer/loading oligomer may include any one of a number of non-palindromic restriction sites with a corresponding “sticky end” available for ligation with another DNA duplex that has been “cut” with the same restriction endonuclease.

Reagent component 2 (or second reagent): A duplex DNA containing a hairpin structure at one end and a “sticky end” for a specified restriction endonuclease at the other end of the duplex. This reagent also contains an acridine residue creating a nick in the phosphodiester sugar backbone of this duplex molecule.

Reagent component 3 (or third reagent): A target DNA duplex for sequencing that contains two different restriction sites, one at each end. One end is complimentary to the two “sticky ends” in the blocking oligomer/loading oligomer duplex and the other end is complimentary to the hairpin containing DNA duplex.

In a preferred embodiment, only a single step is used to create the sequencing reagent: Reagents 1, 2, and 3 at appropriate molar ratios including 1:1:1, are mixed in appropriate buffers, cofactors, and DNA ligase. This results in the ligation of the three DNA duplexes together. This mixture is then ready for direct addition to the Nanopore-Polymerase DNA sequencing device. Synthesis of this construct can also be accomplished by other methods that also result in the ligated construct that comprises the features disclosed herein.

### Using the Invention

A simple method and necessary reagents to prepare and sequence DNA of any length with a Nanopore-Polymerase sequencing device, while meeting the properties of a Nanopore-Polymerase sequencing device is disclosed. The invention comprises (1) generation of a recognizable signal indicating DNA to be sequenced with an attached DNA polymerase (DNAP, molecular motor) are both properly oriented at the atomic/molecular level to start the sequencing process; (2) generation of recognizable signals for steps within the process of DNA sequencing with the Nanopore-Polymerase Sequencing Device. This includes information on the initiation of polynucleotide sequencing, progress and completion of the sequencing process, and (3) proper positioning of the polymerase in relation to the nanopore and DNA in order to initiate enzymatic polymerase driven movement of the sequenced strand through the nanopore for the purpose reading DNA sequence.

The method and reagents of the invention is for sequencing a polynucleotide. A polynucleotide, such as a nucleic acid, is a macromolecule comprising two or more nucleotides. The polynucleotide or nucleic acid may comprise any combination of any nucleotides. The nucleotides can be naturally occurring or artificial. The nucleotide can be oxidized or methylated. A nucleotide typically contains a nucleobase, a sugar and at least one phosphate group. The nucleobase is typically heterocyclic. Nucleobases include, but are not limited to, purines and pyrimidines and more specifically adenine, guanine, thymine, uracil and cytosine. The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The nucleotides are preferably selected from AMP, TMP, GMP, CMP, UMP, dAMP, dTMP, dGMP or dCMP.

A nucleotide may contain a sugar and at least one phosphate group (that is, lack a nucleobase).

The polynucleotide may be single stranded or double stranded. At least a portion of the polynucleotide is preferably double stranded.

The polynucleotide can be a nucleic acid, such as deoxyribonucleic acid (DNA) or ribonucleic acid (RNA). The target polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains.

The whole or only part of the target nucleic acid sequence may be sequenced using this method. The target polynucleotide can be any length. For example, the polynucleotide can be at least 10, at least 50, at least 100, at least 150, at least 200, at least 250, at least 300, at least 400 or at least 500 nucleotide pairs in length. The polynucleotide can be 1000 or more nucleotide pairs, 5000 or more nucleotide pairs in length or 100000 or more nucleotide pairs in length.

The target polynucleotide is present in any suitable sample. The invention is typically carried out on a sample that is known to contain or suspected to contain the target polynucleotide. Alternatively, the invention may be carried out on a sample to confirm the identity of one or more target polynucleotides whose presence in the sample is known or expected.

The sample may be a biological sample. The invention may be carried out in vitro on a sample obtained from or extracted from any organism or microorganism. The organism or microorganism is typically prokaryotic or eukaryotic and typically belongs to one the five kingdoms: plantae, animalia, fungi, monera and protista. The invention may be carried out in vitro on a sample obtained from or extracted from any virus. The sample is preferably a fluid sample. The sample typically comprises a body fluid of the patient. The sample may be urine, lymph, saliva, mucus or amniotic fluid but is preferably blood, plasma or serum. Typically, the sample is human in origin, but alternatively it may be from another mammal animal such as from commercially farmed animals such as horses, cattle, sheep or pigs or may alternatively be pets such as cats or dogs. Alternatively a sample of plant origin is typically obtained from a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa, cotton.

The sample may be a non-biological sample. The non-biological sample is preferably a fluid sample. Examples of a non-biological sample include surgical fluids, water such as drinking water, seawater or river water, and reagents for laboratory tests.

The sample is typically processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

A transmembrane pore is a structure that permits hydrated ions driven by an applied potential to flow from one side of the membrane to the other side of the membrane.

Any membrane may be used in accordance with the invention. Suitable membranes are well known in the art. The membrane is preferably an amphiphilic layer. An amphiphilic layer is a layer formed from amphiphilic molecules, such as phospholipids, which have both hydrophilic and lipophilic properties.

The membrane is preferably a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

Methods for forming lipid bilayers are known in the art. Suitable methods are disclosed in the Example. Lipid bilayers are commonly formed by the method of Montal and Mueller (Proc. Natl. Acad. Sci. USA, 1972; 69: 3561-3566), in which a lipid monolayer is carried on aqueous solution/air interface past either side of an aperture which is perpendicular to that interface.

The method of Montal & Mueller (1972) is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

In a preferred embodiment, the lipid bilayer is formed as described in International Application No. PCT/GB08/004127 (published as WO 2009/077734). Advantageously in this method, the lipid bilayer is formed from dried lipids. In a most preferred embodiment, the lipid bilayer is formed across an opening as described in WO2009/077734 (PCT/GB08/004127).

In another preferred embodiment, the membrane is a solid state layer. A solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si3N4, Al203, and SiO, organic and inorganic polymers such as polyamide, plastics such as TEFLON or elastomers such as two-component addition-cure silicone rubber, and glasses. The solid-state layer may be formed from graphene. Suitable graphene layers are disclosed in International Application No. PCT/US2008/010637 (published as WO 2009/035647). The solid-state layer may be formed from silicon, silicon nitride, or graphene. The solid-state layer may further comprise a solid-state pore or a plurality of such pores. The solid-state layer or pore may further comprise a linker group compound that is attached by covalent bond. A DNA Polymerase may be attached to a solid-state layer or solid state pore using a suitable linker group.

The method is typically carried out using (i) an artificial bilayer comprising a pore, (ii) an isolated, naturally-occurring lipid bilayer comprising a pore, or (iii) a cell having a pore inserted therein. The method is preferably carried out using an artificial lipid bilayer. The bilayer may comprise other transmembrane and/or intramembrane proteins as well as other molecules in addition to the pore. Suitable apparatus and conditions are discussed below with reference to the sequencing embodiments of the invention. The method of the invention is typically carried out in vitro.

The transmembrane pore is preferably a transmembrane protein pore. A transmembrane protein pore is a polypeptide or a collection of polypeptides that permits hydrated ions, such as analyte, to flow from one side of a membrane to the other side of the membrane. In the present invention, the transmembrane protein pore is capable of forming a pore that permits hydrated ions driven by an applied potential to flow from one side of the membrane to the other. The transmembrane protein pore preferably permits analyte such as nucleotides to flow from one side of the membrane, such as a lipid bilayer, to the other. The transmembrane protein pore allows a polynucleotide, such as DNA or RNA, to be moved through the pore.

The transmembrane protein pore may be a monomer or an oligomer. The pore is preferably made up of several repeating subunits, such as 6, 7 or 8 subunits. The pore is more preferably a heptameric or octameric pore.

The transmembrane protein pore typically comprises a barrel or channel through which the ions may flow. The subunits of the pore typically surround a central axis and contribute strands to a transmembrane β barrel or channel or a transmembrane α-helix bundle or channel.

The barrel or channel of the transmembrane protein pore typically comprises amino acids that facilitate interaction with analyte, such as nucleotides, polynucleotides or nucleic acids. These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and a outer membrane proteins, such as WZA and ClyA toxin. The transmembrane pore may be derived from, for example, Msp or from α-hemolysin (α-HL).

Single-channel thin film devices and methods for using the same are provided. The subject devices comprise cis and trans chambers connected by an electrical communication means. At the cis end of the electrical communication means is a horizontal conical aperture sealed with a thin film that includes a single nanopore or channel. The devices further include a means for applying an electric field between the cis and trans chambers. The subject devices find use in applications in which the ionic current through a nanopore or channel is monitored, where such applications include the characterization of naturally occurring ion channels, the characterization of polymeric compounds, and the like.

In particular, the invention provides a novel system comprising a nanopore positioned between the cis and trans chambers and a DNA polymerase isolated from a mesophile, a halophile, or an extreme halophile microorganism. In one preferred embodiment, the DNA polymerase isolated from the mesophile prokaryote is phi29 DNAP protein. In another preferred embodiment, the DNA polymerase comprises a 5′-3′ polymerase and a 3′-5′ exonuclease. In a more preferred embodiment, the halophile microorganism is an extreme halophile microorganism. In the alternative, the DNA polymerase is isolated from a virus that can infect a mesophile, a halophile, or an extreme halophile microorganism.

The DNA polymerase may be active in low salt concentrations, for example less than 0.5M salt, or under high-salt concentrations, for example, at least about 0.5 M, at least about 0.6 M, at least about 1 M, at least about 1.5 M, at least about 2 M, at least about 2.5 M, at least about 3 M, at least about 3.5 M, at least about 4 M, at least about 4.5 M, at least about 5 M, at least about 5.5 M, and at saturation.

The invention also provides a DNA polymerase that may also be active for significantly longer time than that of a Klenow (exo-) fragment under similar conditions. In one example, the DNA polymerase of the invention can be active for up to 40 seconds compared with a few milliseconds using Klenow (exo-) fragment. This ˜10,000-fold increase in activity is clearly an unexpectedly superior result that would not have been predicted by the prior art in any combination, including T7 DNA polymerase which is known to be highly processive in bulk phase when bound to thioredoxin but which rapidly dissociates when captured on a nanopore (Olasagasti, F.; Lieberman, K. R.; Benner, S.; Cherf, G. M.; Dahl, J. M.; Deamer, D. W.; Akeson, M., Nat. Nanotechnol. 2010, 5(11): 789-806, doi:10.1038/nnano.2010.177. The invention provides a DNA polymerase that may be active for 40 seconds, for 60 seconds, for 120 seconds, for 5 minutes, for 10 minutes, for 15 minutes, for 20 minutes, for 30 minutes, for 45 minutes, for 60 minutes, for 1.5 hours, for 2 hours, for 4 hours, for 8 hours, for 12 hours, for 16 hours, for 20 hours, for 24 hours, for several days, or for several weeks, including more than one month, or even indefinitely. One additional advantage of the invention is that in some instances or circumstances, it is not necessary to provide a step of waiting for a reaction to occur.

In one embodiment, the DNA polymerase activity results in a terminal cascade, a series of discrete ionic current steps.

### Exemplary Uses of the Invention

(1) A nanopore device comprising the reagents disclosed herein can be used to monitor the turnover of enzymes such as exonucleases and polymerases, which have important applications in DNA sequencing.

(2) A nanopore device comprising the reagents disclosed herein can function as a biosensor to monitor the interaction between soluble substances such as enzyme substrates or signaling molecules. Examples include blood components such as glucose, uric acid and urea, hormones such as steroids and cytokines, and pharmaceutical agents that exert their function by binding to receptor molecules.

(3) A nanopore device comprising the reagents disclosed herein can monitor in real time the function of important biological structures such as ribosomes, and perform this operation with a single functional unit.

### Manufacture of Single Channel Thin Film Devices

Single-channel thin film devices and methods for using the same are provided. The subject devices comprise a mixed-signal semiconductor wafer, at least one electrochemical layer, the electrochemical layer comprising a semiconductor material, such as silicon dioxide or the like, wherein the semiconductor material further comprises a surface modifier, such as a hydrocarbon, wherein the electrochemical layer defines a plurality of orifices, the orifices comprising a chamber and a neck and wherein the chamber of the orifices co-localize with a first metal composition of the mixed-signal semiconductor wafer, wherein a portion of the orifice is plugged with a second metal, for example, silver, wherein the second metal is in electronic communication with the first metal, and wherein the orifice further comprises a thin film, such as a phospholipid bilayer, the thin film forming a solvent-impermeable seal at the neck of the orifice, the thin film further comprising a pore, and wherein the orifice encloses an aqueous phase and a gas phase. In a preferred embodiment the metallization layer comprises a metal, or metal alloy, such as, but not limited to, nickel, gold, copper, and aluminum.

Pores for use in accordance with the invention can be β-barrel pores or α-helix bundle pores. β-barrel pores comprise a barrel or channel that is formed from β-sheets. Suitable β-barrel pores include, but are not limited to, β-toxins, such as α-hemolysin and leukocidins, and outer membrane proteins/porins of bacteria, such as Mycobacterium smegmatis porin A (MspA), MspB, MspC, MspD, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and outer membrane proteins, such as E. coli Wza and ClyA toxin. Other useful pore proteins may include the NNN-RRK mutant of the MspA monomer that includes the following mutations: D90N, D91N, D93N, D118R, D134R and E139K.

Methods are known in the art for inserting subunits into membranes, such as lipid bilayers. For example, subunits may be suspended in a purified form in a solution containing a lipid bilayer such that it diffuses to the lipid bilayer and is inserted by binding to the lipid bilayer and assembling into a functional state. Alternatively, subunits may be directly inserted into the membrane using the “pick and place” method described in M. A. Holden, H. Bayley. J. Am. Chem. Soc. 2005, 127, 6502-6503 and International Application No. PCT/GB2006/001057 (published as WO 2006/100484).

The concentration of pore molecule or channel molecule is sufficient to form a single channel in any of the thin films or bilayers in approximately, for example, fifteen minutes. The time to form such channels can be for example, between one-half minute and one hour, for example, about one-half minute, one minute, two minutes, three minutes, four minutes, five minutes, seven minutes, ten minutes, fifteen minutes, twenty minutes, twenty five minutes, thirty minutes, thirty five minutes, forty minutes, forty five minutes, fifty minutes, fifty five minutes, sixty minutes, or any time therebetween. The time for formation can be altered by an operator by several factors or parameters, for example, increasing or decreasing the ambient or incubation temperature, increasing or decreasing the concentration of salt in second solution or first solution, placing a potential difference between the first solution and the second solution that attracts the pore or channel molecule towards the thin film or bilayer, or other methods know to those of skill in the art. The finite state machine can detect and/or sense formation of a single channel in its corresponding bilayer by reacting to the flow of current (ions) through the circuit, the circuit comprising the macroscopic electrode, the second solution, the single nanopore or channel molecule, first solution, and the metal electrode for any given array element.

Formation of biological channels is a stochastic process. Once a single channel has formed in a given array element bilayer, it is preferred that the chance that a second channel so forming therein is reduced or preferably, eliminated. The probability of second channel insertion can be modulated with applied potential, that is potential difference, across the bilayer. Upon sensing a single channel, the finite state machine adjusts the potential on the metal electrode to decrease the possibility of second channel insertion into the same bilayer.

In an alternative embodiment, each array element may comprise a gold electrode surrounding the orifice. This gold electrode may serve to activate chemical reagents using reduction or oxidation reactions and that can act specifically at the location of a specific orifice.

The nanopore system can be created using state-of-the-art commercially available 65 nm process technology, for example from Taiwan Semiconductor Manufacturing Company, Taiwan). A 600×600 array of nanopores can perform 360,000 biochemical reaction and detection/sensing steps at a rate of 1000 Hz. This may enable sequencing of polynucleotides, for example, to proceed at a rate of 360 million baser per second per 1 cm×1 cm die cut from the semiconductor wafer.

Exemplary means for applying an electric field between the cis- and trans-chambers are, for example, electrodes comprising an immersed anode and an immersed cathode, that are connected to a voltage source. Such electrodes can be made from, for example silver chloride, or any other compound having similar physical and/or chemical properties.

Devices that can be used to carry out the methods of the instant invention are described in for example, U.S. Pat. No. 5,795,782, U.S. Pat. No. 6,015,714, U.S. Pat. No. 6,267,872, U.S. Pat. No. 6,746,594, U.S. Pat. No. 6,428,959, and U.S. Pat. No. 6,617,113, each of which is hereby incorporated by reference in their entirety.

### Detection

Time-dependent transport properties of the nanopore aperture may be measured by any suitable technique. The transport properties may be a function of the medium used to transport the polynucleotide, solutes (for example, ions) in the liquid, the polynucleotide (for example, chemical structure of the monomers), or labels on the polynucleotide. Exemplary transport properties include current, conductance, resistance, capacitance, charge, concentration, optical properties (for example, fluorescence and Raman scattering), and chemical structure. Desirably, the transport property is current.

Exemplary means for detecting the current between the cis and the trans chambers have been described in WO 00/79257, U.S. Pat. Nos. 6,46,594, 6,673 6,673,615, 6,627,067, 6,464,842, 6,362,002, 6,267,872, 6,015,714, and 5,795,782 and U.S. Publication Nos. 2004/0121525, 2003/0104428, and 2003/0104428, and can include, but are not limited to, electrodes directly associated with the channel or pore at or near the pore aperture, electrodes placed within the cis and the trans chambers, ad insulated glass micro-electrodes. The electrodes may be capable of, but not limited to, detecting ionic current differences across the two chambers or electron tunneling currents across the pore aperture or channel aperture. In another embodiment, the transport property is electron flow across the diameter of the aperture, which may be monitored by electrodes disposed adjacent to or abutting on the nanopore circumference. Such electrodes can be attached to an Axopatch 200B amplifier for amplifying a signal.

Applications and/or uses of the invention disclosed herein may include, but not be limited to the following:
1. Assay of relative or absolute gene expression levels as indicated by mRNA, rRNA, and tRNA. This includes natural, mutated, and pathogenic nucleic acids and polynucleotides.
2. Assay of allelic expressions.
3. Haplotype assays and phasing of multiple SNPs within chromosomes.
4. Assay of DNA methylation state.
5. Assay of mRNA alternate splicing and level of splice variants.
6. Assay of RNA transport.
7. Assay of protein-nucleic acid complexes in mRNA, rRNA, and DNA.
8. Assay of the presence of microbe or viral content in food and environmental samples via DNA, rRNA, or mRNA.
9. Identification of microbe or viral content in food and environmental samples via DNA, rRNA, or mRNA.
10. Identification of pathologies via DNA, rRNA, or mRNA in plants, human, microbes, and animals.
11. Assay of nucleic acids in medical diagnosis.
12. Quantitative nuclear run off assays.
13. Assay of gene rearrangements at DNA and RNA levels, including, but not limited to those found in immune responses.
14. Assay of gene transfer in microbes, viruses and mitochondria.
15. Assay of genetic evolution.
16. Forensic assays.
17. Paternity assays.
18. Genealogical assays.

Polynucleotides homologous to other polynucleotides may be identified by hybridization to each other under stringent or under highly stringent conditions. Single-stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited above.

Encompassed by the invention are polynucleotide sequences that are capable of hybridizing to polynucleotides and fragments thereof under various conditions of stringency (for example, in Wahl and Berger (1987) Methods Enzymol. 152: 399-407, and Kimmel (1987) Methods Enzymol. 152: 507-511). Estimates of homology are provided by either DNA-DNA or DNA-RNA hybridization under conditions of stringency as is well understood by those skilled in the art (Hames and Higgins, Editors (1985) Nucleic Acid Hybridisation: A Practical Approach, IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

### Characterization and Uses of the Invention

### Sequencing

In one embodiment, the invention may be used to perform sequence analysis of polynucleotides. The analyses have an advantage over the prior art and the current art in that a single analysis may be performed at a single site, thereby resulting in considerable cost savings for reagents, substrates, reporter molecules, and the like. Of additional import is the rapidity of the sequencing reaction and the signal generated, thereby resulting in an improvement over the prior art.

Other methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the HYDRA microdispenser (Robbins Scientific, Sunnyvale Calif.), MICROLAB 2200 system (Hamilton, Reno Nev.), and the DNA ENGINE thermal cycler (PTC200; MJ Research, Watertown Mass.). Machines used for sequencing include the ABI PRISM 3700, 377 or 373 DNA sequencing systems (PE Biosystems), the MEGABACE 1000 DNA sequencing system (Amersham Pharmacia Biotech), and the like. The sequences may be analyzed using a variety of algorithms that are well known in the art and described in Ausubel et al. (1997; Short Protocols in Molecular Biology, John Wiley & Sons, New York N.Y., unit 7.7) and Meyers (1995; Molecular Biology and Biotechnology, Wiley VCH, New York N.Y., pp. 856-853).

Shotgun sequencing is used to generate more sequence from cloned inserts derived from multiple sources. Shotgun sequencing methods are well known in the art and use thermostable DNA polymerases, heat-labile DNA polymerases, and primers chosen from representative regions flanking the polynucleotide molecules of interest. Incomplete assembled sequences are inspected for identity using various algorithms or programs such as CONSED (Gordon (1998) Genome Res. 8: 195-202) that are well known in the art. Contaminating sequences including vector or chimeric sequences or deleted sequences can be removed or restored, respectively, organizing the incomplete assembled sequences into finished sequences.

### Extension of a Polynucleotide Sequence

The sequences of the invention may be extended using various PCR-based methods known in the art. For example, the XL-PCR kit (PE Biosystems), nested primers, and commercially available cDNA or genomic DNA libraries may be used to extend the polynucleotide sequence. For all PCR-based methods, primers may be designed using commercially available software, such as OLIGO 4.06 primer analysis software (National Biosciences, Plymouth Minn.) to be about 22 to 30 nucleotides in length, to have a GC content of about 50% or more, and to anneal to a target molecule at temperatures from about 55° C. to about 68° C. When extending a sequence to recover regulatory elements, it is preferable to use genomic, rather than cDNA libraries.

Use of Polynucleotides with the Invention

### Labeling of Molecules for Assay

A wide variety of labels and conjugation techniques are known by those skilled in the art and may be used in various nucleic acid, amino acid, and antibody assays. Synthesis of labeled molecules may be achieved using Promega (Madison Wis.) or Amersham Pharmacia Biotech kits for incorporation of a labeled nucleotide such as ³²P-dCTP, Cy3-dCTP or Cy5-dCTP or amino acid such as ³⁵S-methionine. Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes, Eugene Oreg.).

### Diagnostics

The polynucleotides, fragments, oligonucleotides, complementary RNA and DNA molecules, and PNAs may be used to detect and quantify altered gene expression, absence/presence versus excess, expression of mRNAs or to monitor mRNA levels during therapeutic intervention. Conditions, diseases or disorders associated with altered expression include idiopathic pulmonary arterial hypertension, secondary pulmonary hypertension, a cell proliferative disorder, particularly anaplastic oligodendroglioma, astrocytoma, oligoastrocytoma, glioblastoma, meningioma, ganglioneuroma, neuronal neoplasm, multiple sclerosis, Huntington's disease, breast adenocarcinoma, prostate adenocarcinoma, stomach adenocarcinoma, metastasizing neuroendocrine carcinoma, nonproliferative fibrocystic and proliferative fibrocystic breast disease, gallbladder cholecystitis and cholelithiasis, osteoarthritis, and rheumatoid arthritis; acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, benign prostatic hyperplasia, bronchitis, Chediak-Higashi syndrome, cholecystitis, Crohn's disease, atopic dermatitis, dermatomyositis, diabetes mellitus, emphysema, erythroblastosis fetalis, erythema nodosum, atrophic gastritis, glomerulonephritis, Goodpasture's syndrome, gout, chronic granulomatous diseases, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polycystic ovary syndrome, polymyositis, psoriasis, Reiter's syndrome, rheumatoid arthritis, scleroderma, severe combined immunodeficiency disease (SCID), Sjogren's syndrome, systemic anaphylaxis, systemic lupus erythematosus, systemic sclerosis, thrombocytopenic purpura, ulcerative colitis, uveitis, Werner syndrome, hemodialysis, extracorporeal circulation, viral, bacterial, fungal, parasitic, protozoal, and helminthic infection; a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, an ectopic pregnancy, and teratogenesis; cancer of the breast, fibrocystic breast disease, and galactorrhea; a disruption of spermatogenesis, abnormal sperm physiology, benign prostatic hyperplasia, prostatitis, Peyronie's disease, impotence, gynecomastia; actinic keratosis, arteriosclerosis, bursitis, cirrhosis, hepatitis, mixed connective tissue disease (MCTD), myelofibrosis, paroxysmal nocturnal hemoglobinuria, polycythemia vera, primary thrombocythemia, complications of cancer, cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In another aspect, the polynucleotide of the invention.

The polynucleotides, fragments, oligonucleotides, complementary RNA and DNA molecules, and PNAs, or fragments thereof, may be used to detect and quantify altered gene expression; absence, presence, or excess expression of mRNAs; or to monitor mRNA levels during therapeutic intervention. Disorders associated with altered expression include akathesia, Alzheimer's disease, amnesia, amyotrophic lateral sclerosis, ataxias, bipolar disorder, catatonia, cerebral palsy, cerebrovascular disease Creutzfeldt-Jakob disease, dementia, depression, Down's syndrome, tardive dyskinesia, dystonias, epilepsy, Huntington's disease, multiple sclerosis, muscular dystrophy, neuralgias, neurofibromatosis, neuropathies, Parkinson's disease, Pick's disease, retinitis pigmentosa, schizophrenia, seasonal affective disorder, senile dementia, stroke, Tourette's syndrome and cancers including adenocarcinomas, melanomas, and teratocarcinomas, particularly of the brain. These cDNAs can also be utilized as markers of treatment efficacy against the diseases noted above and other brain disorders, conditions, and diseases over a period ranging from several days to months. The diagnostic assay may use hybridization or amplification technology to compare gene expression in a biological sample from a patient to standard samples in order to detect altered gene expression. Qualitative or quantitative methods for this comparison are well known in the art.

The diagnostic assay may use hybridization or amplification technology to compare gene expression in a biological sample from a patient to standard samples in order to detect altered gene expression. Qualitative or quantitative methods for this comparison are well known in the art.

For example, the polynucleotide or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a substantially purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate the level that is observed in a normal subject. The results obtained from successive assays may be used to show the efficacy of treatment over a period ranging from several days to months.

### Purification of Ligand

The polynucleotide or a fragment thereof may be used to purify a ligand from a sample. A method for using a polynucleotide or a fragment thereof to purify a ligand would involve combining the polynucleotide or a fragment thereof with a sample under conditions to allow specific binding, detecting specific binding, recovering the bound protein, and using an appropriate agent to separate the polynucleotide from the purified ligand.

In additional embodiments, the polynucleotides may be used in any molecular biology techniques that have yet to be developed, provided the new techniques rely on properties of polynucleotides that are currently known, including, but not limited to, such properties as the triplet genetic code and specific base pair interactions.

### Composition of the DNA Polymerase

The invention also contemplates variants of the processory DNA polymerase. Such variants may have increased or decreased binding affinity for DNA. Such variants may also have increased or decreased rates of reaction. For example, in the KF, the reactive tyrosine residue may be substituted by, for example, tryptophan.

Amino acid substitutions may be made to an peptide sequence, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, |
| | | | neutral |
| Asp | polar, hydrophilic, charged (−) | Pro | hydrophobic, neutral |
| Glu | polar, hydrophilic, charged (−) | Gln | polar, hydrophilic, |
| | | | neutral |
| Phe | aromatic, hydrophobic, neutral | Arg | polar, hydrophilic, |
| | | | charged (+) |
| Gly | aliphatic, neutral | Ser | polar, hydrophilic, |
| | | | neutral |
| His | aromatic, polar, hydrophilic, | Thr | polar, hydrophilic, |
| | charged (+) | | neutral |
| Ile | aliphatic, hydrophobic, neutral | Val | aliphatic, hydrophobic, |
| | | | neutral |
| Lys | polar, hydrophilic, charged(+) | Trp | aromatic, hydrophobic, |
| | | | neutral |
| Leu | aliphatic, hydrophobic, neutral | Tyr | aromatic, polar, |
| | | | hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Conservative substitutions are those in which at least one residue in the amino acid sequence has been removed and a different residue inserted in its place. Such substitutions generally are made in accordance with the Table 3 when it is desired to maintain the activity of the protein. Table 2 shows amino acids which can be substituted for an amino acid in a protein and which are typically regarded as conservative substitutions.

TABLE 3

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Similar substitutions are those in which at least one residue in the amino acid sequence has been removed and a different residue inserted in its place. Such substitutions generally are made in accordance with the Table 4 when it is desired to maintain the activity of the protein. Table 4 shows amino acids which can be substituted for an amino acid in a protein and which are typically regarded as structural and functional substitutions. For example, a residue in column 1 of Table 4 may be substituted with a residue in column 2; in addition, a residue in column 2 of Table 4 may be substituted with the residue of column 1.

TABLE 4

| Residue | Similar Substitutions |
|---|---|
| Ala | Ser; Thr; Gly; Val; Leu; Ile |
| Arg | Lys; His; Gly |
| Asn | Gln; His; Gly; Ser; Thr |
| Asp | Glu, Ser; Thr |
| Gln | Asn; Ala |
| Cyc | Ser; Gly |
| Glu | Asp |
| Gly | Pro; Arg |
| His | Asn; Gln; Tyr; Phe; Lys; Arg |
| Ile | Ala; Leu; Val; Gly; Met |
| Leu | Ala; Ile; Val; Gly; Met |
| Lys | Arg; His; Gln; Gly; Pro |
| Met | Leu; Ile; Phe |
| Phe | Met; Leu; Tyr; Trp; His; Val; Ala |
| Ser | Thr; Gly; Asp; Ala; Val; Ile; His |
| Thr | Ser; Val; Ala; Gly |
| Trp | Tyr; Phe; His |
| Tyr | Trp; Phe; His |
| Val | Ala; Ile; Leu; Gly; Thr; Ser; Glu |

Substitutions that are less conservative than those in Table 2 can be selected by picking residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histidyl, is substituted for (or by) an electronegative residue, for example, glutamyl or aspartyl; or (d) a residue having a bulky side chain, for example, phenylalanine, is substituted for (or by) one not having a side chain, for example, glycine.

The transmembrane protein pore is also preferably derived from α-hemolysin (α-HL). The wild type α-HL pore is formed of seven identical monomers or subunits (that is, it is heptameric).

These methods are possible because transmembrane protein pores can be used to differentiate nucleotides of similar structure on the basis of the different effects they have on the current passing through the pore. Individual nucleotides can be identified at the single molecule level from their current amplitude when they interact with the pore. The nucleotide is present in the pore if the current flows through the pore in a manner specific for the nucleotide (that is, if a distinctive current associated with the nucleotide is detected flowing through the pore). Successive identification of the nucleotides in a target polynucleotide allows the sequence of the polynucleotide to be determined. As discussed above, this is Strand Sequencing.

During the interaction between a nucleotide in the single stranded polynucleotide and the pore, the nucleotide affects the current flowing through the pore in a manner specific for that nucleotide. For example, a particular nucleotide will reduce the current flowing through the pore for a particular mean time period and to a particular extent. In other words, the current flowing through the pore is distinctive for a particular nucleotide. Control experiments may be carried out to determine the effect a particular nucleotide has on the current flowing through the pore. Results from carrying out the method of the invention on a test sample can then be compared with those derived from such a control experiment in order to determine the sequence of the target polynucleotide.

The sequencing methods may be carried out using any apparatus that is suitable for investigating a membrane/pore system in which a pore is inserted into a membrane. The method may be carried out using any apparatus that is suitable for transmembrane pore sensing. For example, the apparatus comprises a chamber comprising an aqueous solution and a barrier that separates the chamber into two sections. The barrier has an aperture in which the membrane containing the pore is formed.

The sequencing methods may be carried out using the apparatus described in International Application No. PCT/GB08/000562.

The methods of the invention involve measuring the current passing through the pore during interaction with the nucleotide(s). Therefore the apparatus also comprises an electrical circuit capable of applying a potential and measuring an electrical signal across the membrane and pore. The methods may be carried out using a patch clamp or a voltage clamp. The methods preferably involve the use of a voltage clamp.

The sequencing methods of the invention involve the measuring of a current passing through the pore during interaction with the nucleotide. Suitable conditions for measuring ionic currents through transmembrane protein pores are known in the art and disclosed in the Example. The method is typically carried out with a voltage applied across the membrane and pore. The voltage used is typically from −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 160 mV to 240 mV. It is possible to increase discrimination between different nucleotides by a pore by using an increased applied potential.

The sequencing methods are typically carried out in the presence of any alkali metal chloride salt. In the exemplary apparatus discussed above, the salt is present in the aqueous solution in the chamber. Potassium chloride (KCl), sodium chloride (NaCl) or caesium chloride (CsCl) is typically used. KCl is preferred. The salt concentration is typically from 0.1 to 2.5M, from 0.3 to 1.9M, from 0.5 to 1.8M, from 0.7 to 1.7M, from 0.9 to 1.6M or from 1M to 1.4M. The salt concentration is preferably from 150 mM to 1M. In some alternative embodiments, it may be desirable to include salt at saturating concentrations. Phi29 DNA polymerase surprisingly works under high salt concentrations. The salt concentration is preferably at least 0.3M, such as at least 0.4M or 0.5 M. High salt concentrations provide a high signal to noise ratio and allow for currents indicative of the presence of a nucleotide to be identified against the background of normal current fluctuations. Lower salt concentrations may be used if nucleotide detection is carried out in the presence of an enzyme.

The methods are typically carried out in the presence of a buffer. In the exemplary apparatus discussed above, the buffer is present in the aqueous solution in the chamber. Any buffer may be used in the method of the invention. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

As mentioned above, good nucleotide discrimination can be achieved at low salt concentrations if the temperature is increased. In addition to increasing the solution temperature, there are a number of other strategies that can be employed to increase the conductance of the solution, while maintaining conditions that are suitable for enzyme activity. One such strategy is to use the lipid bilayer to divide two different concentrations of salt solution, a low salt concentration of salt on the enzyme side and a higher concentration on the opposite side. One example of this approach is to use 200 mM of KCl on the cis side of the membrane and 500 mM KCl in the trans chamber. At these conditions, the conductance through the pore is expected to be roughly equivalent to 400 mM KCl under normal conditions, and the enzyme only experiences 200 mM if placed on the cis side. Another possible benefit of using asymmetric salt conditions is the osmotic gradient induced across the pore. This net flow of water could be used to pull nucleotides into the pore for detection. A similar effect can be achieved using a neutral osmolyte, such as sucrose, glycerol or PEG. Another possibility is to use a solution with relatively low levels of KCl and rely on an additional charge carrying species that is less disruptive to enzyme activity.

The target polynucleotide being analysed can be combined with known protecting chemistries to protect the polynucleotide from being acted upon by the binding protein while in the bulk solution. The pore can then be used to remove the protecting chemistry. This can be achieved either by using protecting groups that are unhybridised by the pore, binding protein or enzyme under an applied potential (WO 2008/124107) or by using protecting chemistries that are removed by the binding protein or enzyme when held in close proximity to the pore (J. Am. Chem. Soc., 2010, 132(50): 17961-17972).

### Kits

The present invention also provides kits for sequencing a target polynucleotide. The kits comprise (a) a pore, (b) a reagent, and (c) a DNA polymerase. Any of the embodiments discussed above with reference to the sequencing method of the invention equally apply to the kits.

The kit may further comprise the components of a membrane, such as the phospholipids needed to form a lipid bilayer.

The kits of the invention may additionally comprise one or more other reagents or instruments that enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides, a membrane as defined above or voltage or patch clamp apparatus. Reagents may be present in the kit in a dry state such that a fluid sample resuspends the reagents. The kit may also, optionally, comprise instructions to enable the kit to be used in the method of the invention or details regarding which patients the method may be used for. The kit may, optionally, comprise nucleotides.

### Apparatus

The invention also provides an apparatus for sequencing a target polynucleotide. The apparatus comprises a plurality of pores, a reagent, and a plurality of DNA polymerases. The apparatus preferably further comprises instructions for carrying out the sequencing method of the invention. The apparatus may be any conventional apparatus for polynucleotide analysis, such as an array or a chip. Any of the embodiments discussed above with reference to the methods of the invention are equally applicable to the apparatus of the invention.

The apparatus is preferably set up to carry out the sequencing method of the invention.

The apparatus preferably comprises:
a. a sensor device that is capable of supporting the membrane and plurality of pores and being operable to perform polynucleotide sequencing using the pores and proteins;
b. at least one reservoir for holding material for performing the sequencing; a fluidics system configured to controllably supply material from the at least one reservoir to the sensor device; and
c. a plurality of containers for receiving respective samples, the fluidics system being configured to supply the samples selectively from the containers to the sensor device.
   The apparatus may be any of those described in International Application No. PCT/GB08/004127 (published as WO 2009/077734), PCT/GB10/000789 (published as WO 2010/122293), International Application No. PCT/GB 10/002206 (not yet published) or International Patent Application No. PCT/US99/25679 (published as WO 00/28312).

The invention will be more readily understood by reference to the following examples, which are included merely for purposes of illustration of certain aspects and embodiments of the present invention and not as limitations.

### EXAMPLES

Herein are described several examples to demonstrate the capability of measuring macromolecules and polyanions or polycations.

### Example I

### Construction of the Blocking Oligomer

The blocking oligomer includes the following features in order 5′ to 3′-a) restriction endonuclease site R, a variable length of DNA strand denoted by ‘>>>>>>>>>>>>>>>>’ (consisting of the bases adenine, guanine, cytosine, and thymidine), and a seven base tail consisting of abasic residues denoted by ‘X’. In addition, other nucleotides may be used, for example, uracil, and modified bases.

An example is provided below.
5′R>>>>>>>>>>>>XXXXXXX

### Example II

### Construction of the Loading Oligomer

The loading oligomer includes the following features in order 5′ to 3′

(a) 35-50 base tail to be threaded through the nanopore to load the nanopore prior to sequencing denoted by ‘ . . . ’,

(b) a viable length of abasic residues including five abasic residues denoted by ‘XXXXX’,

(c) a length of DNA bases complimentary to and equal in length to the DNA base sequences between the restriction endonuclease site and seven abasic residues of the blocking oligomer denoted by ‘>>>>>>>>>>>>>>>>’, and

(d) a restriction endonuclease recognition site ‘R’. An example is provided below.

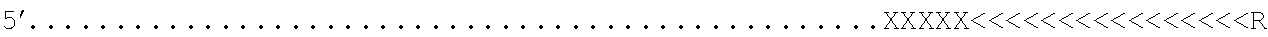

### Example III

### Construction of the Hairpin Containing Duplex with “Sticky Ends” and a Nick in the Phosphate Sugar Backbone

The hairpin containing duplex includes the following features in order 5′ to 3′
(a) a restriction endonuclease site ‘R’,
(b) a short 5-10 base denoted by ‘>>>>>>’,
(c) a hairpin with a 6-10 base complimentary stem, one or more acridine containing residues for the purpose of creating a nick in the phosphodiester sugar backbone; denoted by ‘Ξ’,
(d) a short 5-10 base segment complementary to the 5-10 base segment on the other side of the hairpin stem denoted by ‘>>>>>>’, and a restriction endonuclease site denoted by ‘R’. An example is provided below.
   TT
   T T
   NN
   NN
   NN
   NN
   NN
   NN
   ΞN Acridine containing residue
   >< creating a nick in
   >< phosphodiester sugar backbone
   ><
   ><
   ><
   RR Restriction endonuclease site (“sticky end”)

### Example IV

### Construction of Target DNA Sequence with “Sticky Ends” for Sequencing

The target DNA strand selected for sequencing has two different restriction endonuclease “sticky ends” matching those on the annealed blocking oligomer/loading oligomer and the hairpin containing oligomer. These DNA duplexes can be constructed by any number of standard molecular biology techniques for placing “sticky ends” on a duplex DNA molecule. These include use of restriction endonucleases to cleave DNA duplexes, shearing of DNA and blunt end ligation of linkers. An example is diagramed below where ‘R’ denotes “sticky ends” for restriction endonuclease sites and the sequence of “periods” ‘ . . . ’ denotes target double strand DNA to be sequenced.

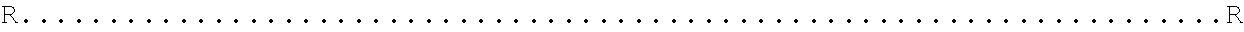

### Example V

### Preparation of Double Strand DNA Reagent for DNA Sequencing

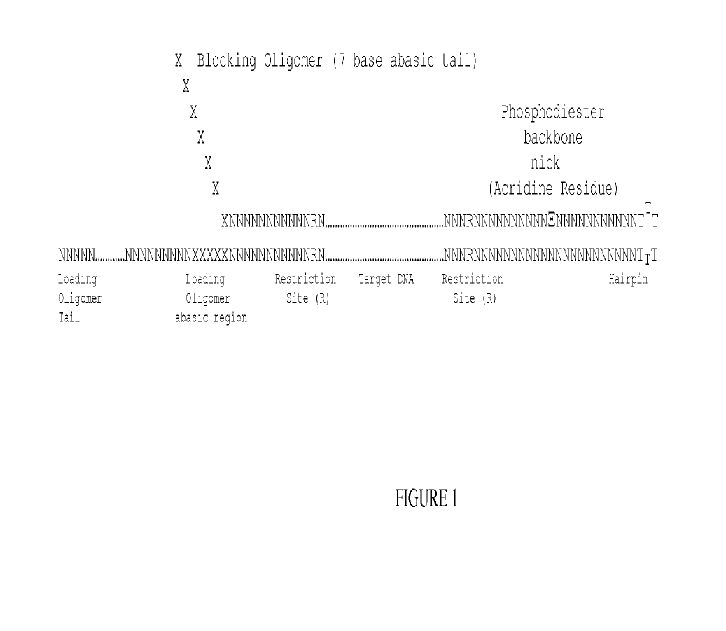

The blocking oligomer was annealed with the loading oligomer by heating a solution containing both oligomers to 95° C. and slow cooling to 4° C. The hairpin containing duplex was prepared by heating a solution containing the hairpin oligo to 95° C. and slow cooling to room temperature, followed by addition of the short 5-10 base oligomer containing the acridine residue. The target DNA to be sequenced was then ligated to the hairpin duplex and the annealed blocking oligomer/loading oligomer duplex using the restriction endonuclease sites and a DNA ligase. Alternative approaches to creating this construct involving the Gibson reaction and other approaches also exist. At this point this mixture was ready for loading on the nanopore-polymerase device for sequencing. FIG. 1 illustrates the ligated DNA sequencing reagent structure.

### Example VI

### Results

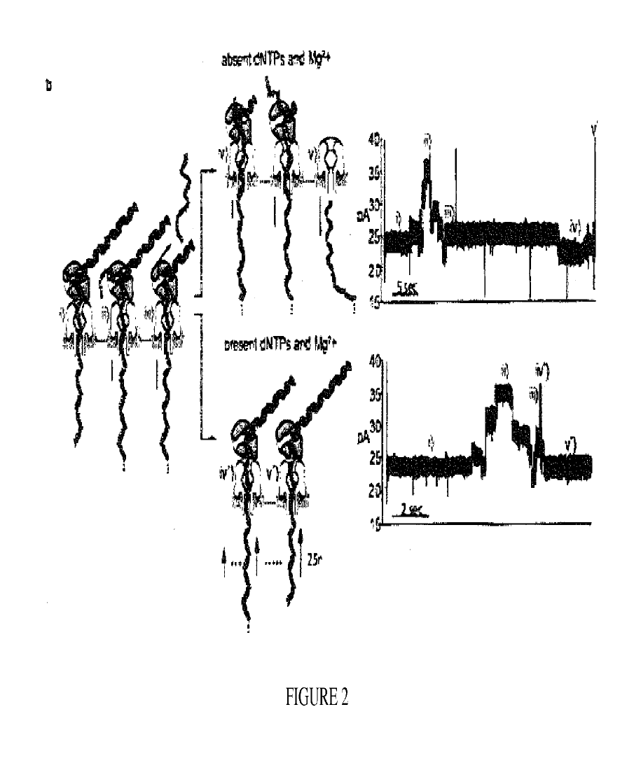

Laboratory data were collected using a DNA reagent as described above and are shown on FIG. 2. Reading and re-reading individual DNA templates on the nanopore using pre-loaded Φ29 DNA polymerase.

Substrate for Φ29 DNAP-catalyzed DNA replication consists of a 94-mer template strand with 5-abasic dNMPs (Xs) at positions +25 to +29 from the tr=0 templating position, a 23-mer primer strand and a 35-mer blocking oligomer strand (red line with two covalently attached acridine residues (orange bars) and an 8-mer unbound tail. An abasic dNMP and C3 spacer (CPG) (S) on the template and blocking oligomer 3′-terminal protect against 3′-5′ exonucleolytic digestion by Φ29 DNAP. (b) Capture and processing of the DNA template on the nanopore. Wild type Φ29 DNAP and the DNA construct in (a) are added to the nanopore chamber. Φ29 DNAP binds up the DNA template in bulk phase but cannot excise or elongate the primer with the blocking oligomer bound. (i) Capture of the Φ29DNAP-DNA complex initially place the 5 abasics (red circles) on the cis side of the nanopore. This gives a current of 24 pA. (ii) The 180 mV applied voltage forces non-catalytic unzipping of the blocking oligomer/template duplex, which causes a rise then fall in the ionic current (peak at 35 pA) as the 5 abasics traverse the limiting aperture of the nanopore. (iii) Further unzipping removes the blocking oligomer and places Φ29 DNAP at the primer/template junction. The 5 abasics are now on the trans side of the nanopore, giving a 23 pA current. In the absence of dNTPs and Mg+2 (top panel) the applied voltage now unzips and removes the DNA primer (iv) leading to dissociation of the DNA template from the nanopore (v). Alternatively in the presence of 100 μM dNTPs and 10 mM Mg+2 (bottom panel) Φ29 DNAP processively replicates 25 DNA bases, causing the DNA template to reverse direction in the nanopore. The 5 abasics traverses the nanopore from the trans side to the cis side, retracing the 35 pA current peak (IV′). Finally the abasic block reaches the polymerase active site and replication stalls (V′).

### Example VII

### Other Enzyme Studies

The FPGA/FSM nanopore system can also be used for other enzyme studies. Applying voltage ramps upon capture of DNA/enzyme complexes can produce data to calculate bond energy landscapes using voltage force spectroscopy. Also, DNA's interaction with the pore can be characterized using feedback control of the applied voltage. Regulation of enzyme catalysis can be by achieved applying tension to DNA occupying the pore, counteracting the enzymes processive force.

### Example VIII

### Isolation of Genomic DNA

Blood samples (2-3 ml) are collected from patients via the pulmonary catheter and stored in EDTA-containing tubes at −80° C. until use. Genomic DNA is extracted from the blood samples using a DNA isolation kit according to the manufacturer's instruction (PUREGENE, Gentra Systems, Minneapolis Minn.). DNA purity is measured as the ratio of the absorbance at 260 and 280 nm (1 cm lightpath; A₂₆₀/A₂₈₀) measured with a Beckman spectrophotometer.

### Example IX

### Identification of SNPs

A region of a gene from a patient's DNA sample is amplified by PCR using the primers specifically designed for the region. The PCR products are sequenced using methods as disclosed above. SNPs identified in the sequence traces are verified using Phred/Phrap/Consed software and compared with known SNPs deposited in the NCBI SNP databank.

### Example X

### cDNA Library Construction

A cDNA library is constructed using RNA isolated from mammalian tissue. The frozen tissue is homogenized and lysed using a POLYTRON homogenizer (Brinkmann Instruments, Westbury N.J.) in guanidinium isothiocyanate solution. The lysates are centrifuged over a 5.7 M CsCl cushion using a SW28 rotor in an L8-70M Ultracentrifuge (Beckman Coulter, Fullerton Calif.) for 18 hours at 25,000 rpm at ambient temperature. The RNA is extracted with acid phenol, pH 4.7, precipitated using 0.3 M sodium acetate and 2.5 volumes of ethanol, resuspended in RNAse-free water, and treated with DNAse at 37° C. RNA extraction and precipitation are repeated as before. The mRNA is isolated with the OLIGOTEX kit (Qiagen, Chatsworth Calif.) and used to construct the cDNA library.

The mRNA is handled according to the recommended protocols in the SUPERSCRIPT plasmid system (Invitrogen). The cDNAs are fractionated on a SEPHAROSE CL4B column (APB), and those cDNAs exceeding 400 bp are ligated into an expression plasmid. The plasmid is subsequently transformed into DH5αa competent cells (Invitrogen).

### Example XI

### Labeling of Probes and Hybridization Analyses

Nucleic acids are isolated from a biological source and applied to a substrate for standard hybridization protocols by one of the following methods. A mixture of target nucleic acids, a restriction digest of genomic DNA, is fractionated by electrophoresis through an 0.7% agarose gel in 1×TAE [Tris-acetate-ethylenediamine tetraacetic acid (EDTA)] running buffer and transferred to a nylon membrane by capillary transfer using 20× saline sodium citrate (SSC). Alternatively, the target nucleic acids are individually ligated to a vector and inserted into bacterial host cells to form a library. Target nucleic acids are arranged on a substrate by one of the following methods. In the first method, bacterial cells containing individual clones are robotically picked and arranged on a nylon membrane. The membrane is placed on bacterial growth medium, LB agar containing carbenicillin, and incubated at 37° C. for 16 hours. Bacterial colonies are denatured, neutralized, and digested with proteinase K. Nylon membranes are exposed to UV irradiation in a STRATALINKER UV-crosslinker (Stratagene) to cross-link DNA to the membrane.

In the second method, target nucleic acids are amplified from bacterial vectors by thirty cycles of PCR using primers complementary to vector sequences flanking the insert. Amplified target nucleic acids are purified using SEPHACRYL-400 beads (Amersham Pharmacia Biotech). Purified target nucleic acids are robotically arrayed onto a glass microscope slide (Corning Science Products, Corning N.Y.). The slide is previously coated with 0.05% aminopropyl silane (Sigma-Aldrich, St. Louis Mo.) and cured at 110° C. The arrayed glass slide (microarray) is exposed to UV irradiation in a STRATALINKER UV-crosslinker (Stratagene).

cDNA probes are made from mRNA templates. Five micrograms of mRNA is mixed with 1 μg random primer (Life Technologies), incubated at 70° C. for 10 minutes, and lyophilized. The lyophilized sample is resuspended in 50 μl of 1× first strand buffer (cDNA Synthesis systems; Life Technologies) containing a dNTP mix, [α-³²P]dCTP, dithiothreitol, and MMLV reverse transcriptase (Stratagene), and incubated at 42° C. for 1-2 hours. After incubation, the probe is diluted with 42 μl dH₂O, heated to 95° C. for 3 minutes, and cooled on ice. mRNA in the probe is removed by alkaline degradation. The probe is neutralized, and degraded mRNA and unincorporated nucleotides are removed using a PROBEQUANT G-50 MicroColumn (Amersham Pharmacia Biotech). Probes can be labeled with fluorescent markers, Cy3-dCTP or Cy5-dCTP (Amersham Pharmacia Biotech), in place of the radionucleotide, [³²P]dCTP.

Hybridization is carried out at 65° C. in a hybridization buffer containing 0.5 M sodium phosphate (pH 7.2), 7% SDS, and 1 mM EDTA. After the substrate is incubated in hybridization buffer at 65° C. for at least 2 hours, the buffer is replaced with 10 ml of fresh buffer containing the probes. After incubation at 65° C. for 18 hours, the hybridization buffer is removed, and the substrate is washed sequentially under increasingly stringent conditions, up to 40 mM sodium phosphate, 1% SDS, 1 mM EDTA at 65° C. To detect signal produced by a radiolabeled probe hybridized on a membrane, the substrate is exposed to a PHOSPHORIMAGER cassette (Amersham Pharmacia Biotech), and the image is analyzed using IMAGEQUANT data analysis software (Amersham Pharmacia Biotech). To detect signals produced by a fluorescent probe hybridized on a microarray, the substrate is examined by confocal laser microscopy, and images are collected and analyzed using gene expression analysis software.

### Example XII

### Complementary Polynucleotides

Molecules complementary to the polynucleotide, or a fragment thereof, are used to detect, decrease, or inhibit gene expression. Although use of oligonucleotides comprising from about 15 to about 30 base pairs is described, the same procedure is used with larger or smaller fragments or their derivatives (for example, peptide nucleic acids, PNAs). Oligonucleotides are designed using OLIGO 4.06 primer analysis software (National Biosciences). To inhibit transcription by preventing a transcription factor binding to a promoter, a complementary oligonucleotide is designed to bind to the most unique 5′ sequence, most preferably between about 500 to 10 nucleotides before the initiation codon of the open reading frame. To inhibit translation, a complementary oligonucleotide is designed to prevent ribosomal binding to the mRNA encoding the mammalian protein.

### Example XIII

### Production of Specific Antibodies

A conjugate comprising a complex of polynucleotide and a binding protein thereof is purified using polyacrylamide gel electrophoresis and used to immunize mice or rabbits. Antibodies are produced using the protocols below. Rabbits are immunized with the complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

### Example XIV

### Screening Molecules for Specific Binding with the Polynucleotide or Protein Conjugate

The polynucleotide, or fragments thereof, are labeled with ³²P-dCTP, Cy3-dCTP, or Cy5-dCTP (Amersham Pharmacia Biotech), or with BIODIPY or FITC (Molecular Probes, Eugene Oreg.), respectively. Similarly, the conjugate comprising a complex of polynucleotide and a binding protein thereof can be labeled with radionucleide or fluorescent probes. Libraries of candidate molecules or compounds previously arranged on a substrate are incubated in the presence of labeled polynucleotide or protein. After incubation under conditions for either a polynucleotide or amino acid molecule, the substrate is washed, and any position on the substrate retaining label, which indicates specific binding or complex formation, is assayed, and the ligand is identified. Data obtained using different concentrations of the polynucleotide or protein are used to calculate affinity between the labeled polynucleotide or protein and the bound molecule.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein.

The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

We claim:

1. A reagent for determining the nucleotide sequence of a target polynucleotide in a sample, the reagent comprising a first reagent component, a second reagent component, a third reagent component, and a DNA modifying enzyme, (i) wherein the first reagent component comprises a 1^{st} polynucleotide partial duplex, the 1^{st} polynucleotide partial duplex comprising a polynucleotide duplex and a first polynucleotide single strand and a second polynucleotide single strand, the polynucleotide duplex comprising a blocking oligomer and a loading oligomer, wherein a portion of the blocking oligomer comprises a nucleotide sequence that is the complement of a portion of the loading oligomer, wherein a portion of the blocking oligomer comprises a nucleotide sequence that is not the complement of a portion of the loading oligomer, wherein the blocking oligomer is annealed to the loading oligomer between the complementary portion of the blocking oligomer and the complementary portion of the loading oligomer thereby creating a proximal portion of the polynucleotide partial duplex, whereby the portion of the blocking oligomer that is not the complement of the loading oligomer is the first single strand and the portion of the loading oligomer that is not the complement of the blocking oligomer is the second single strand, and wherein the blocking oligomer first single strand and the loading oligomer second single strand comprise a distal portion of the polynucleotide partial duplex; (ii) wherein the second reagent component comprises a 2^{nd} polynucleotide partial duplex, the 2^{nd} polynucleotide partial duplex comprising a single polynucleotide, wherein a first portion of the single polynucleotide is substantially the complement of a second portion of the polynucleotide, and wherein the 2^{nd} polynucleotide partial duplex comprises the first portion of the single polynucleotide is annealed to the second portion of the polynucleotide thereby creating a hairpin structure, the hairpin structure comprising a hairpin loop, a hairpin stem, the hairpin stem further comprising at least one acridine nucleotide residue, and third portion of the polynucleotide that is not annealed to either portion of the polynucleotide, the third portion further comprising a restriction endonuclease site; (iii) wherein the third reagent component comprises the target polynucleotide, the target polynucleotide substantially comprising a double strand polynucleotide and a first end and a second end, wherein the first end comprises a first strand that is the complement of the blocking oligomer and a second strand that is the complement of the loading oligomer, wherein the second end comprises a polynucleotide sequence that is the complement of the hairpin structure; and wherein the first and second and third reagent components are at an equimolar ratio.

2. The reagent of claim 1, wherein the DNA modifying enzyme is a DNA ligase.

3. A system for determining the nucleotide sequence of a polynucleotide in a sample, the system comprising an electrical source, an anode, a cathode, a cis chamber, a trans chamber, wherein the cis and the trans chambers are separated by a thin film, the thin film having a plurality of apertures, wherein each aperture is between about 0.25 nm and about 4 nm in diameter, a conducting solvent, a processive DNA modifying enzyme, a plurality of dNTP molecules, a metal ion co-factor, and the reagent of claim 1.

4. An apparatus for determining the nucleotide sequence of a polynucleotide in a sample, the apparatus comprising an electrical source, an anode, a cathode, a cis chamber, a trans chamber, wherein the cis and the trans chambers are separated by a thin film, the thin film having a plurality of apertures (pores), wherein each aperture (pore) is between about 0.25 nm and about 4 nm in diameter, a conducting solvent, a processive DNA modifying enzyme, a plurality of dNTP molecules, a metal ion co-factor, and the reagent of claim 1.

5. A device for determining the nucleotide sequence of a polynucleotide in a sample, the device comprising an electrical source, an anode, a cathode, a cis chamber, a trans chamber, wherein the cis and the trans chambers are separated by a thin film, the thin film having a plurality of apertures (pores), wherein each aperture (pore) is between about 0.25 nm and about 4 nm in diameter, a conducting solvent, a processive DNA modifying enzyme, a plurality of dNTP molecules, a metal ion co-factor, and the reagent of claim 1.

6. A method for determining the nucleotide sequence of a polynucleotide in a sample, the method comprising the steps of: providing two separate adjacent chambers comprising a liquid medium, an interface between the two chambers, the interface having an aperture so dimensioned as to allow sequential monomer-by-monomer passage from the cis-side of the channel to the trans-side of the channel of only one polynucleotide strand at a time; providing the reagent of claim 1; introducing the reagent of claim 1 into one of the two chambers; allowing the processive DNA-modifying enzyme to bind to the polynucleotide; applying a potential difference between the two chambers, thereby creating a first polarity, the first polarity causing the single-stranded portion of the polynucleotide to transpose through the aperture to the trans-side; introducing the enzyme into the same chamber; allowing the enzyme to bind to the polynucleotide; measuring the electrical current through the channel thereby detecting a nucleotide base in the polynucleotide; decreasing the potential difference a first time; allowing the single-stranded portion of the polynucleotide to transpose through the aperture; measuring the change in electrical current; increasing the potential difference; measuring the electrical current through the channel, thereby detecting a particular nucleotide base positioned at the aperture; repeating any one of the steps, thereby determining the nucleotide sequence of the polynucleotide.

7. The method of claim 6, wherein the method further comprises a step of adding at least one species of ddNTP molecule.

8. The system, apparatus, device, or method of any of claim 3, 4, or 5, wherein the system, apparatus, device, or method further comprises at least one species of ddNTP molecule.

9. The system, apparatus, device, or method of any of claim 3, 4, or 5, wherein the concentration of one dNTP molecule is at least two orders of magnitude lower than the concentration of the other dNTP molecules.

10. The system, apparatus, device, or method of any of claim 3, 4, or 5, wherein the system, apparatus, device, or method further comprises an ammeter.

11. The system, apparatus, device, or method of any of claim 3, 4, or 5, wherein the aperture diameter is about 2 nm.

12. The system, apparatus, device, or method of any of claim 3, 4, or 5, wherein the conducting solvent is an aqueous solvent.

13. The system, apparatus, device, or method of any of claim 3, 4, or 5, wherein the conducting solvent is a non-aqueous solvent.

14. The system, apparatus, device, or method of any of claim 3, 4, or 5, wherein the processive DNA modifying enzyme is a DNA polymerase.

15. The system, apparatus, device, or method of any of claim 3, 4, or 5, wherein the processive DNA modifying enzyme is selected from the group consisting of phi29 DNA polymerase, T7 DNA polymerase, His 1 DNA polymerase, and His 2 DNA polymerase, Bacillus phage M2 DNA polymerase, Streptococcus phage CP1 DNA polymerase, enterobacter phage PRD 1 DNA polymerase, and variants thereof.

16. The system, apparatus, device, or method of any of claim 3, 4, or 5, wherein the variant of the DNA modifying enzyme has at least 85% amino acid identity with the wild-type DNA modifying enzyme.

17. Use of the reagent of claim 1 to control the movement of a target polynucleotide through a pore.

18. The use of the reagent to control the movement of a target polynucleotide through a pore of claim 17, wherein the use of the reagent further comprises (i) providing the reagent of claim 1 adjacent to a pore in a DNA sequencing device, (ii) positioning a DNA Polymerase in relationship to a nanopore and a target polynucleotide to be sequenced thereby allowing the DNA polymerase to function as a molecular motor and thereby providing single base resolution movement of the target polynucleotide through the pore in the DNA sequencing device, (iii) whereby the position of the DNA Polymerase in relationship to a nanopore and a target polynucleotide generates a diagnostic signal in the DNA sequencing device thereby indicating the target polynucleotide and DNA polymerase are in the correct position to begin sequencing the target polynucleotide in the DNA sequencing device, (iv) generating signals confirming steps in the sequencing of individual bases, including the beginning, the middle and then end of the target polynucleotide, and (v) repeating (i), (ii), (iii), and (iv).

19. A kit for sequencing a target polynucleotide comprising (a) a pore and (b) the reagent of claim 1.

20. An analysis apparatus for sequencing target polynucleotides in a sample, comprising a plurality of pores and the reagent of claim 1.

21. The analysis apparatus according to claim 20, wherein the analysis apparatus comprises:
a sensor device that is capable of supporting the plurality of pores and being operable to perform polynucleotide sequencing using the pores and polymerases;
at least one reservoir for holding material for performing the sequencing;
a fluidics system configured to controllably supply material from the at least one reservoir to the sensor device; and
a plurality of containers for receiving respective samples, the fluidics system being configured to supply the samples selectively from the containers to the sensor device.
